(19) United States
(12) Patent Application Publication
(10) Pub. No.: US 2017/0247437 A1
(43) Pub. Date: Aug. 31, 2017

(54) RSPO1 BINDING AGENTS AND USES THEREOF

(71) Applicant: OncoMed Pharmaceuticals, Inc., Redwood City, CA (US)

(72) Inventor: Austin L. GURNEY, San Francisco, CA (US)

(21) Appl. No.: 15/503,928

(22) PCT Filed: Aug. 14, 2015
(86) PCT No.: PCT/US15/45210

§ 371 Date: Feb. 14, 2017

Related U.S. Application Data
(60) Provisional application No. 62/037,880, filed on Aug. 15, 2014.

(51) Int. Cl. C07K 16/18; A61K 39/395; C12Q 1/68; A61K 45/06

(52) U.S. Cl. CPC C07K 16/18; A61K 45/06; A61K 39/39558; C12Q 1/6886; C07K 2317/565; C07K 2317/24; C07K 2317/31; A61K 2039/507

(57) ABSTRACT

The present invention relates to RSPO-binding agents, particularly antibodies that specifically bind human RSPO1. Also described are methods of treating cancer and other diseases, comprising administering a therapeutically effect amount of an agent or antibody of the present invention to a patient in need thereof.

### CROSS-REFERENCE TO RELATED APPLICATIONS

This application claims the priority benefit of U.S. Provisional Application No. 62/037,880, filed Aug. 15, 2014, which is hereby incorporated by reference herein in its entirety.

### FIELD OF THE INVENTION

The field of this invention generally relates to antibodies and other agents that bind R-Spondin protein 1 (RSPO1). The invention also generally relates to methods of producing the antibodies and methods of using the antibodies for the treatment of diseases such as cancer.

### BACKGROUND OF THE INVENTION

The R-Spondin (RSPO) family of proteins is conserved among vertebrates and comprises four members, RSPO1, RSPO2, RSPO3 and RSPO4. These proteins have been referred to by a variety of names, including roof plate-specific spondins, hPWTSR (hRSPO3), THS2D (RSPO3), Cristin 1-4, and Futrin 1-4. The RSPOs are small secreted proteins that overall share approximately 40-60% sequence homology and domain organization. All RSPO proteins contain two furin-like cysteine-rich domains at the N-terminus followed by a thrombospondin domain and a basic charged C-terminal tail (Kim et al., 2006, Cell Cycle, 5:23-26).

Studies have shown that RSPO proteins have a role during vertebrate development (Kamata et al., 2004, Biochim. Biophys Acta, 1676:51-62) and in Xenopus myogenesis (Kazanskaya et al., 2004, Dev. Cell, 7:525-534). RSPO1 has also been shown to function as a potent mitogen for gastrointestinal epithelial cells (Kim et al., 2005, Science, 309:1256-1259). RSPO proteins are known to activate β-catenin signaling similar to Wnt signaling, however the relationship between RSPO proteins and Wnt signaling is still being investigated. It has been reported that RSPO proteins possess a positive modulatory activity on Wnt ligands (Nam et al., 2006, JBC 281:13247-57). This study also reported that

RSPO proteins could function as Frizzled8 and LRP6 receptor ligands and induce β-catenin signaling (Nam et al., 2006, JBC 281:13247-57). Recent studies have identified an interaction between RSPO proteins and LGR (leucine-rich repeat containing, G protein-coupler receptor) proteins, such as LGRS (U.S. Pat. Nos. 8,158,758 and 8,158,757), and these data present an alternative pathway for the activation of β-catenin signaling.

The Wnt signaling pathway has been identified as a potential target for cancer therapy. The Wnt signaling pathway is one of several critical regulators of embryonic pattern formation, post-embryonic tissue maintenance, and stem cell biology. More specifically, Wnt signaling plays an important role in the generation of cell polarity and cell fate specification including self-renewal by stem cell populations. Unregulated activation of the Wnt pathway is associated with numerous human cancers where it is believed the activation can alter the developmental fate of cells. The activation of the Wnt pathway may maintain tumor cells in an undifferentiated state and/or lead to uncontrolled proliferation. Thus, carcinogenesis can proceed by overtaking homeostatic mechanisms which control normal development and tissue repair (reviewed in Reya & Clevers, 2005, Nature, 434:843-50; Beachy et al., 2004, Nature, 432:324-31).

The Wnt signaling pathway was first elucidated in the Drosophila developmental mutant wingless (wg) and from the murine proto-oncogene int-1, now Wnt1 (Nusse & Varmus, 1982, Cell, 31:99-109; Van Ooyen & Nusse, 1984, Cell, 39:233-40; Cabrera et al., 1987, Cell, 50:659-63; Rijsewijk et al., 1987, Cell, 50:649-57). Wnt genes encode secreted lipid-modified glycoproteins of which 19 have been identified in mammals. These secreted ligands activate a receptor complex consisting of a Frizzled (FZD) receptor family member and low-density lipoprotein (LDL) receptor-related protein 5 or 6 (LRPS/6). The FZD receptors are seven transmembrane domain proteins of the G-protein coupled receptor (GPCR) superfamily and contain a large extracellular N-terminal ligand binding domain with 10 conserved cysteines, known as a cysteine-rich domain (CRD) or Fri domain. There are ten human FZD receptors, FZD1, FZD2, FZD3, FZD4, FZDS, FZD6, FZD7, FZD8, FZD9, and FZD10. Different FZD CRDs have different binding affinities for specific Wnt proteins (Wu & Nusse, 2002, J. Biol. Chem., 277:41762-9), and FZD receptors have been grouped into those that activate the canonical β-catenin pathway and those that activate non-canonical pathways (Miller et al., 1999, Oncogene, 18:7860-72).

A role for Wnt signaling in cancer was first uncovered with the identification of Wnt1 (originally inti) as an oncogene in mammary tumors transformed by the nearby insertion of a murine virus (Nusse & Varmus, 1982, Cell, 31:99-109). Additional evidence for the role of Wnt signaling in breast cancer has since accumulated. For instance, transgenic over-expression of β-catenin in the mammary glands results in hyperplasias and adenocarcinomas (Imbert et al., 2001, J. Cell Biol., 153:555-68; Michaelson & Leder, 2001, Oncogene, 20:5093-9) whereas loss of Wnt signaling disrupts normal mammary gland development (Tepera et al., 2003, J. Cell Sci., 116:1137-49; Hatsell et al., 2003, J. Mammary Gland Biol. Neoplasia, 8:145-58). In human breast cancer, β-catenin accumulation implicates activated Wnt signaling in over 50% of carcinomas, and though specific mutations have not been identified, up-regulation of Frizzled receptor expression has been observed (Brennan & Brown, 2004, J. Mammary Gland Biol. Neoplasia, 9:119-31; Malovanovic et al., 2004, Int. J. Oncol., 25:1337-42).

Activation of the Wnt pathway is also associated with colorectal cancer. Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Mutations have also been identified in other Wnt pathway components including Axin and β-catenin. Individual adenomas are clonal outgrowths of epithelial cells containing a second inactivated allele, and the large number of FAP adenomas inevitably results in the development of adenocarcinomas through additional mutations in oncogenes and/or tumor suppressor genes. Furthermore, activation of the Wnt signaling pathway, including loss-of-function mutations in APC and stabilizing mutations in β-catenin, can induce hyperplastic development and tumor growth in mouse models (Oshima et al., 1997, Cancer Res., 57:1644-9; Harada et al., 1999, EMBO J., 18:5931-42).

Similar to breast cancer and colon cancer, melanoma often has constitutive activation of the Wnt pathway, as indicated by the nuclear accumulation of β-catenin. Activation of the Wnt/β-catenin pathway in some melanoma tumors and cell lines is due to modifications in pathway components, such as APC, ICAT, LEF1 and β-catenin (see e.g., Lame et al. 2006, Frontiers Biosci., 11:733-742). However, there are conflicting reports in the literature as to the exact role of Wnt/β-catenin signaling in melanoma. For example, one study found that elevated levels of nuclear β-catenin correlated with improved survival from melanoma, and that activated Wnt/β-catenin signaling was associated with decreased cell proliferation (Chien et al., 2009, PNAS, 106:1193-1198).

The focus of cancer drug research is shifting toward targeted therapies aimed at genes, proteins, and pathways involved in human cancer. There is a need for new agents targeting signaling pathways and new combinations of agents that target multiple pathways that could provide therapeutic benefit for cancer patients.

BRIEF SUMMARY OF THE INVENTION

The present invention provides binding agents, such as antibodies, that bind RSPO1 proteins, as well as compositions, such as pharmaceutical compositions, comprising the binding agents. In certain embodiments, the RSPO1-binding agents are novel polypeptides, such as antibodies, antibody fragments, and other polypeptides related to such antibodies. In some embodiments, the RSPO1-binding agents are antibody variants with desirable and/or improved properties from a production and/or purification perspective. In some embodiments, the RSPO1-binding agents are antibody variants with desirable properties such as improved solubility or improved stability. In some embodiments, the RSPO1-binding agents are antibody variants with desirable and/or improved properties from a therapeutic perspective. The invention further provides methods of inhibiting the growth of a tumor by administering the RSPO1-binding agents to a subject with a tumor. The invention further provides methods of treating cancer by administering the RSPO1-binding agents to a subject in need thereof In some embodiments, the methods of treating cancer or inhibiting tumor growth comprise targeting cancer stem cells with the RSPO1-binding agents. In certain embodiments, the methods comprise reducing the frequency of cancer stem cells in a tumor, reducing the number of cancer stem cells in a tumor, reducing the tumorigenicity of a tumor, and/or reducing the tumorigenicity of a tumor by reducing the number or frequency of cancer stem cells in the tumor.

In one aspect, the invention provides a binding agent, such as an antibody, that specifically binds human RSPO1. In certain embodiments, the RSPO1-binding agent binds within amino acids 21-263 of human RSPO1 (SEQ ID NO:1). In certain embodiments, the RSPO1-binding agent binds within amino acids 34-135 of human RSPO1 (SEQ ID NO:1). In certain embodiments, the RSPO1-binding agent binds within amino acids 34-85 of human RSPO1 (SEQ ID NO:1). In certain embodiments, the RSPO1-binding agent binds within amino acids 91-135 of human RSPO1 (SEQ ID NO:1). In some embodiments, the RSPO1-binding agent (e.g., an antibody) specifically binds at least one other human RSPO selected from the group consisting of RSPO2, RSPO3, and RSPO4. In some embodiments, the RSPO1-binding agent or antibody when binding human RSPO1 (i) modulates β-catenin activity, (ii) is an antagonist of β-catenin signaling, (iii) inhibits β-catenin signaling, and/or (iv) inhibits activation of β-catenin. In some embodiments, the RSPO1-binding agent when binding RSPO1 inhibits RSPO1 signaling. In some embodiments, the RSPO1-binding agent inhibits or interferes with binding of RSPO1 to one or more LGR proteins (e.g., LGR4, LGRS, and/or LGR6). In some embodiments, the RSPO1-binding agent inhibits binding of RSPO1 to LGRS.

In certain embodiments of each of the aforementioned aspects and embodiments, as well as other aspects and embodiments described herein, the RSPO1-binding agent is an antibody. In certain embodiments, the antibody is a monoclonal antibody. In certain embodiments, the antibody is a humanized antibody. In certain embodiments, the antibody binds human RSPO1. In certain embodiments, the antibody binds human RSPO1 and mouse RSPO1. In certain embodiments, the antibody binds human RSPO1 with a K_{D} of less than 1 nM and mouse RSPO1 with a K_{D} of less than 1 nM.

In certain embodiments, the RSPO1-binding agent is an antibody which comprises a heavy chain CDR1 comprising TGYTMH (SEQ ID NO:6), a heavy chain CDR2 comprising GINPNNGGTTYNQNFKG (SEQ ID NO:7), and a heavy chain CDR3 comprising KEFSDGYYFFAY

(SEQ ID NO:8). In some embodiments, the antibody further comprises a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11). In certain embodiments, the RSPO1-binding agent is an antibody which comprises a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11).

In certain embodiments, the RSPO1-binding agent is an antibody which comprises: (a) a heavy chain variable region having at least 90% sequence identity to SEQ ID NO:32, and/or (b) a light chain variable region having at least 90% sequence identity to SEQ ID NO:33. In certain embodiments, the RSPO1-binding agent is an antibody that comprises: (a) a heavy chain variable region having at least 95% sequence identity to SEQ ID NO:32, and/or (b) a light chain variable region having at least 95% sequence identity to SEQ ID NO:33. In certain embodiments, the RSPO1-binding agent is an antibody that comprises: (a) a heavy chain variable region having at least 91%, 92%, 93%, 94%, 95%, 96%, 97%, 98% or 99% sequence identity to SEQ ID NO:32, and/or (b) a light chain variable region having at least 91%, 92%, 93%, 94%, 95%, 96%, 97%, 98% or 99% sequence identity to SEQ ID NO:33.

In certain embodiments, the RSPO1-binding agent is an antibody which comprises a heavy chain having at least 90% sequence identity to SEQ ID NO:35, and a light chain having at least 90% sequence identity to SEQ ID NO:37. In certain embodiments, the RSPO1-binding agent is an antibody that comprises a heavy chain having at least 95% sequence identity to SEQ ID NO:35 and a light chain having at least 95% sequence identity to SEQ ID NO:37. In certain embodiments, the RSPO1-binding agent is an antibody that comprises a heavy chain having at least 91%, 92%, 93%, 94%, 95%, 96%, 97%, 98% or 99% sequence identity to SEQ ID NO:35 and a light chain having at least 91%, 92%, 93%, 94%, 95%, 96%, 97%, 98% or 99% sequence identity to SEQ ID NO:37

In some embodiments, the RSPO1-binding agent is a humanized form of antibody 89M5. Monoclonal antibody 89M5 is produced by the hybridoma cell line 89M5 deposited on June 30, 2011 with American Type Culture Collection (ATCC) having deposit no. PTA-11970. In some embodiments, the RSPO1-binding agent is humanized monoclonal antibody h89M5-H8L5. In some embodiments, the RSPO1-binding agent comprises a heavy chain variable region encoded by the plasmid deposited with ATCC on August 15, 2014 and having deposit no. PTA-121494. In some embodiments, the RSPO1-binding agent comprises a light chain variable region encoded by the plasmid deposited with ATCC on

August 15, 2014 and having deposit no. PTA-121495. In some embodiments, the RSPO1-binding agent comprises a heavy chain encoded by the plasmid deposited with ATCC on August 15, 2014 and having deposit no. PTA-121494. In some embodiments, the RSPO1-binding agent comprises a light chain encoded by the plasmid deposited with ATCC on August 15, 2014 and having deposit no. PTA-121495.

In another aspect, the invention provides a binding agent (e.g., an antibody) that competes for specific binding to a human RSPO protein with an antibody of the invention. In some embodiments, the binding agent (e.g., an antibody) competes for specific binding to human RSPO1 with an antibody that comprises a heavy chain variable region comprising SEQ ID NO:32, and a light chain variable region comprising SEQ ID NO:33. In some embodiments, the antibody with which the RSPO1-binding agent competes is h89M5-H8L5. In some embodiments, the binding agent competes for specific binding to

RSPO1 with an antibody of the invention in an in vitro competitive binding assay.

In certain embodiments, the antibody binds the same epitope, or essentially the same epitope, on RSPO1 as an antibody of the invention (e.g., h89M5-H8L5).

In still another aspect, the binding agent is an antibody that binds an epitope on RSPO1 that overlaps with the epitope on RSPO1 bound by an antibody of the invention (e.g., h89M5-H8L5).

In certain embodiments of each of the aforementioned aspects, as well as other aspects and/or embodiments described elsewhere herein, the RSPO1-binding agent or antibody is isolated.

In another aspect, the invention provides a polypeptide comprising SEQ ID NO:32 and/or SEQ ID NO:33. In another aspect, the invention provides a polypeptide comprising SEQ ID NO:34 and/or SEQ ID NO:36. In another aspect, the invention provides a polypeptide comprising SEQ ID NO:35 and/or SEQ ID NO:37. In some embodiments, the polypeptide is isolated. In certain embodiments, the polypeptide is substantially pure. In certain embodiments, the polypeptide is an antibody.

In another aspect, the invention provides isolated polynucleotide molecules comprising a polynucleotide that encodes the antibodies and/or polypeptides of each of the aforementioned aspects, as well as other aspects and/or embodiments described herein. In some embodiments, the polynucleotide comprises a polynucleotide that encodes an amino acid sequence selected from the group consisting of SEQ ID NO:32, SEQ ID NO:33, SEQ ID NO:34, SEQ ID NO:35, SEQ ID NO:36, and SEQ ID NO:37. In some embodiments, the polynucleotide comprises a sequence selected from the group consisting of SEQ ID NO:38, SEQ ID NO:39, SEQ ID NO:40, and SEQ ID NO:41. The invention further provides expression vectors that comprise the polynucleotides, as well as cells that comprise the expression vectors and/or the polynucleotides. In some embodiments, the cell is a hybridoma cell line.

In other aspects, the invention provides methods of inhibiting growth of a tumor, comprising contacting the tumor with an effective amount of a RSPO1-binding agent or antibody, including each of those described herein.

In another aspect, the invention provides a method of inhibiting the growth of a tumor in a subject, comprising administering to the subject a therapeutically effective amount of a RSPO1-binding agent or antibody, including each of those described herein.

In another aspect, the invention provides a method of inhibiting β-catenin signaling in a cell, comprising contacting the cell with an effective amount of a RSPO1-binding agent or antibody, including each of those described herein. In some embodiments, the cell is a tumor cell. In some embodiments, the tumor is a colorectal tumor. In some embodiments, the tumor is an ovarian tumor. In some embodiments, the tumor is a pancreatic tumor. In some embodiments, the tumor is a lung tumor. In some embodiments, the tumor expresses elevated levels of at least one RSPO protein. In some embodiments, the tumor expresses elevated levels of RSPO1. In some embodiments, the tumor expresses elevated levels of RSPO2. In some embodiments, the tumor expresses elevated levels of RSPO3. In certain embodiments, the RSPO1-binding agent inhibits growth of the tumor, for example, by reducing the number and/or frequency of cancer stem cells in the tumor.

In another aspect, the invention provides methods of treating cancer in a subject. In some embodiments, the method comprises administering to a subject a therapeutically effective amount of any of the RSPO1-binding agents or antibodies described herein. In some embodiments, the cancer is pancreatic cancer. In some embodiments, the cancer is colorectal cancer. In some embodiments, the colorectal cancer comprises an inactivating mutation in the adenomatous polyposis coli (APC) gene. In some embodiments, the colorectal cancer does not comprise an inactivating mutation in the APC gene. In some embodiments, the colorectal cancer comprises a wild-type APC gene. In some embodiments, the cancer is ovarian cancer. In some embodiments, the cancer is breast cancer. In some embodiments, the cancer is lung cancer. In some embodiments, the cancer expresses elevated levels of at least one RSPO protein. In some embodiments, the cancer expresses elevated levels of RSPO1. In some embodiments, the cancer is an ovarian cancer that expresses elevated levels of RSPO1.

In another aspect, the invention provides methods of treating a disease in a subject wherein the disease is associated with activation of β-catenin, and/or aberrant β-catenin signaling comprising administering a therapeutically effective amount of a RSPO1-binding agent or antibody, including each of those described herein.

In certain embodiments of each of the aforementioned aspects, as well as other aspects and/or embodiments described elsewhere herein, the treatment methods comprise administering a RSPO1-binding agent in combination with at least one additional therapeutic agent. In some embodiments, the treatment methods comprise administering a RSPO1-binding agent in combination with a second RSPO-binding agent such as a RSPO2-binding agent, a RSPO3-binding agent, and/or a RSPO4-binding agent. In some embodiments, the treatment methods comprise administering a RSPO1-binding agent in combination with a RSPO2-binding agent. In some embodiments, the treatment methods comprise administering a combination of a RSPO1-binding agent, a RSPO2-binding agent, and at least one chemotherapeutic agent.

In certain embodiments of each of the aforementioned aspects, as well as other aspects and/or embodiments described elsewhere herein, the treatment methods further comprise a step of determining the expression level of at least one RSPO protein in the tumor or cancer.

In another aspect, the invention provides a method of identifying a human subject or selecting a human subject for treatment with a RSPO1-binding agent or antibody, including but not limited to, each of those described herein. In some embodiments, the method comprises determining if the subject has a tumor that has an elevated expression level of a specific RSPO (e.g., RSPO1, RSPO2, or RSPO3) as compared to a predetermined expression level of the RSPO protein. In some embodiments, the method comprises identifying a subject for treatment or selecting a subject for treatment if the tumor has an elevated level of RSPO expression. In some embodiments, the method comprises determining if the subject has a tumor that comprises an inactivating mutation in the APC gene. In some embodiments, the method comprises identifying a subject for treatment or selecting a subject for treatment if the tumor comprises an inactivating mutation in the APC gene.

Compositions comprising a RSPO1-binding agent or antibody described herein are further provided. Pharmaceutical compositions comprising a RSPO1-binding agent or antibody described herein and a pharmaceutically acceptable carrier are further provided. Cell lines that produce the RSPO1-binding agents described herein are provided. Methods of treating cancer and/or inhibiting tumor growth in a subject (e.g., a human) comprising administering to the subject an effective amount of a pharmaceutical composition comprising the RSPO1-binding agents are also provided.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTION OF THE FIGURES

FIGS. 1A and 1B. Amino acid sequence of first generation anti-RSPO1 humanized antibody h89M5-H2L2 compared to second generation anti-RSPO humanized antibody h89M5-H8L5. FIG. 1A—Heavy chain amino acid sequence with CDRs indicated. FIG. 1B—Light chain amino acid sequence with CDRs indicated.

FIG. 2. FACS analysis of humanized anti-RSPO1 antibodies. FACS analyses of first generation anti-RSPO1 humanized antibody h89M5-H2L2 and second generation anti-RSPO1 humanized antibody h89M5-H8L5. Serial dilutions of each antibody were tested. Relative antibody binding is shown on the y-axis and expression of the FLAG-RSPO1 furin-CD4TM-GFP fusion protein is indicated on the x-axis.

FIG. 3. Size exclusive chromatography analysis of humanized anti-RSPO1 antibodies. The bottom profile is the first generation anti-RSPO1 antibody h89M5-H2L2. The middle profile is the second generation anti-RSPO1 antibody h89M5-H8L5. The top profile is MW standards.

### DETAILED DESCRIPTION OF THE INVENTION

The present invention provides novel agents, including, but not limited to polypeptides such as antibodies, that bind RSPO proteins (e.g., human RSPO1, RSPO2, and/or RSPO3). The RSPO1-binding agents include antagonists of β-catenin signaling. Related polypeptides and polynucleotides, compositions comprising the RSPO1-binding agents, and methods of making the RSPO1-binding agents are also provided. Methods of using the novel RSPO1-binding agents, such as methods of inhibiting tumor growth, methods of treating cancer, methods of reducing the frequency of cancer stem cells in a tumor, methods of inhibiting β-catenin signaling, and/or methods of identifying and/or selecting subjects for treatment, are further provided.

### I. Definitions

To facilitate an understanding of the present invention, a number of terms and phrases are defined below.

The terms “antagonist” and “antagonistic” as used herein refer to any molecule that partially or fully blocks, inhibits, reduces or neutralizes a biological activity of a target and/or signaling pathway (e.g., the β-catenin signaling). The term “antagonist” is used herein to include any molecule that partially or fully blocks, inhibits, reduces or neutralizes the activity of a protein (e.g., a RSPO protein). Suitable antagonist molecules specifically include, but are not limited to, antagonist antibodies or antibody fragments.

The terms “modulation” and “modulate” as used herein refer to a change or an alteration in a biological activity. Modulation includes, but is not limited to, stimulating or inhibiting an activity.

Modulation may be an increase or a decrease in activity (e.g., a decrease in RSPO signaling; a decrease in β-catenin signaling), a change in binding characteristics, or any other change in the biological, functional, or immunological properties associated with the activity of a protein, pathway, or other biological point of interest.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies such as bispecific antibodies generated from at least two antibodies, monospecific antibodies, monovalent antibodies, chimeric antibodies, humanized antibodies, human antibodies, fusion proteins comprising an antigen determination portion of an antibody, and any other modified immunoglobulin molecule comprising an antigen recognition site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises an antigen-binding site or epitope-binding site.

The term “variable region” of an antibody refers to the variable region of the antibody light chain, or the variable region of the antibody heavy chain, either alone or in combination. The variable regions of the heavy and light chain each consist of four framework regions (FR) connected by three complementarity determining regions (CDRs), also known as “hypervariable regions”. The CDRs in each chain are held together in close proximity by the framework regions and, generally with the CDRs from the other chain, contribute to the formation of the antigen-binding sites of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen recognition site (antigen-binding site). Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences. Typically, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some instances, the framework region residues of a human immunoglobulin are replaced with the corresponding residues in an antibody from a non-human species. The humanized antibody can be further modified by the substitution of additional residues either in the Fv framework region and/or within the replaced non-human residues to refine and optimize antibody specificity, affinity, and/or binding capability. In general, the humanized antibody will comprise the variable domains containing all or substantially all of the CDRs that correspond to the non-human immunoglobulin whereas all or substantially all of the framework regions are those of a human immunoglobulin sequence. The humanized antibody can also comprise at least a portion of an immunoglobulin constant region or domain (Fc), typically that of a human immunoglobulin.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody wherein the amino acid sequence of the immunoglobulin molecule is derived from two or more species. Typically, the variable region of both the light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human).

The phrase “affinity matured antibody” as used herein refers to an antibody with one or more alterations in one or more CDRs thereof that result in an improvement in the affinity of the antibody for antigen, compared to a parent antibody that does not possess those alterations(s). Preferred affinity matured antibodies will have nanomolar or even picomolar affinities for the target antigen. Affinity matured antibodies are produced by methods known in the art, including but not limited to, heavy chain variable region and light chain variable region domain shuffling, random mutagenesis of CDR and/or framework residues, and site-directed mutagenesis of CDR and/or framework residues.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “selectively binds” or “specifically binds” mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that an antibody binds a protein with a K_{D} of about 0.1 mM or less, but more usually less than about 1 μM. In certain embodiments, “specifically binds” means that an antibody binds a target at times with a K_{D} of at least about 0.1 μM or less, at other times at least about 0.01 μM or less, and at other times at least about 1 nM or less. Because of the sequence identity between homologous proteins in different species, specific binding can include an antibody that recognizes a protein in more than one species (e.g., human RSPO1 and mouse RSPO1). Likewise, because of homology within certain regions of polypeptide sequences of different but related proteins, specific binding can include an antibody (or other polypeptide or binding agent) that recognizes more than one protein (e.g., human RSPO1 and human RSPO2). It is understood that, in certain embodiments, an antibody or binding moiety that specifically binds a first target may or may not specifically bind a second target. As such, “specific binding” does not necessarily require (although it can include) exclusive binding, i.e. binding to a single target. Thus, an antibody may, in certain embodiments, specifically bind more than one target. In certain embodiments, multiple targets may be bound by the same antigen-binding site on the antibody. For example, an antibody may, in certain instances, comprise two identical antigen-binding sites, each of which specifically binds the same epitope on two or more proteins (e.g., RSPO1 and RSPO2). In certain alternative embodiments, an antibody may be bispecific or multispecific and comprise at least two antigen-binding sites with differing specificities. By way of non-limiting example, a bispecific antibody may comprise one antigen-binding site that recognizes an epitope on one protein (e.g., human RSPO1) and further comprise a second, different antigen-binding site that recognizes a different epitope on a second protein. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies, in certain embodiments, the polypeptides can occur as single chains or associated chains.

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by hybridization conditions that: (1) employ low ionic strength and high temperature for washing, for example 15 mM sodium chloride/1.5 mM sodium citrate/0.1% sodium dodecyl sulfate at 50° C.; (2) employ a denaturing agent, such as formamide, for example, 50% (v/v) formamide with 0.1% bovine serum albumin/0 1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5× SSC (0.75 M NaCl, 75 mM sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2× SSC and 50% formamide at 55° C., followed by a high-stringency wash consisting of 0.1× SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value in between. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Preferably, conservative substitutions in the sequences of the polypeptides and antibodies of the invention do not abrogate the binding of the polypeptide or antibody containing the amino acid sequence, to the antigen(s), i.e., the one or more RSPO protein(s) to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions.

The term “metastasis” as used herein refers to the process by which a cancer spreads or transfers from the site of origin to other regions of the body with the development of a similar cancerous lesion at a new location. A “metastatic” or “metastasizing” cell is one that loses adhesive contacts with neighboring cells and migrates via the bloodstream or lymph from the primary site of disease to invade neighboring body structures.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced proliferative or developmental potential; and (3) are capable of symmetric cell division for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to approved or approvable by a regulatory agency of the Federal or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one binding agent (e.g., an antibody) of the present disclosure, and which does not destroy the pharmacological activity thereof and is nontoxic when administered in doses sufficient to deliver a therapeutic effect. In general, those of skill in the art and the FDA consider a pharmaceutically acceptable excipient, carrier, or adjuvant to be an inactive ingredient of any formulation.

The terms “effective amount” or “therapeutically effective amount” or “therapeutic effect” refer to an amount of a binding agent, an antibody, a polypeptide, a polynucleotide, a small organic molecule, or other drug effective to “treat” a disease or disorder in a subject or mammal. In the case of cancer, the therapeutically effective amount of a drug (e.g., an antibody) has a therapeutic effect and as such can reduce the number of cancer cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those already with the disorder; those prone to have the disorder; and those in whom the disorder is to be prevented. In some embodiments, a subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. RSPO1-Binding Agents

The present invention provides agents that bind human RSPO1 proteins. These agents are referred to herein as “RSPO1-binding agents”. In some embodiments, the RSPO1-binding agents are antibodies. In some embodiments, the RSPO1-binding agents are polypeptides. In certain embodiments, the RSPO1-agents specifically bind at least one other human RSPO. In some embodiments, the at least one other human RSPO bound by a RSPO1-binding agent is selected from the group consisting of RSPO2, RSPO3, and RSPO4. The full-length amino acid (aa) sequence for human RSPO1 is known in the art and is provided herein as SEQ ID NO:1.

In certain embodiments, the RSPO1-binding agent is an antibody that specifically binds within amino acids 21-263 of human RSPO1 (SEQ ID NO:1). In certain embodiments, the RSPO1-binding agent is an antibody that specifically binds within amino acids 31-263 of human RSPO1 (SEQ ID NO:1).

In certain embodiments, the RSPO1-binding agent is an antibody that specifically binds within amino acids 34-135 of human RSPO1 (SEQ ID NO:1). In certain embodiments, the RSPO1-binding agent is an antibody that specifically binds within amino acids 34-85 of human RSPO1 (SEQ ID NO:1). In certain embodiments, the RSPO1-binding agent is an antibody that specifically binds within amino acids 91-135 of human RSPO1 (SEQ ID NO:1). In certain embodiments, the RSPO1-binding agent binds within SEQ

ID NO:1. In some embodiments, the RSPO1-binding agent binds within SEQ ID NO:5. In certain embodiments, the RSPO1-binding agent binds a furin-like cysteine-rich domain of RSPO1. In some embodiments, the RSPO1-binding agent binds at least one amino acid within a furin-like cysteine-rich domain of RSPO1. In certain embodiments, the RSPO1-binding agent binds within sequence SEQ ID NO:2 or SEQ ID NO:3. In some embodiments, the RSPO1-binding agent binds the thrombospondin domain of RSPO1. In some embodiments, the RSPO1-binding agent binds at least one amino acid within the thrombospondin domain of RSPO1. In some embodiments, the RSPO1-binding agent binds within SEQ ID NO:4. In some embodiments, the RSPO1-binding agent (e.g., an antibody) specifically binds both human RSPO1 and mouse RSPO1.

In certain embodiments, a RSPO1-binding agent (e.g., an antibody) binds RSPO1 with a dissociation constant (K_{D}) of about 1 μM or less, about 100 nM or less, about 40 nM or less, about 20 nM or less, about 10 nM or less, about 1nM or less, or about 0.1nM or less. In some embodiments, a RSPO1-binding agent binds RSPO1 with a K_{D} of about 1 nM or less. In some embodiments, a RSPO1-binding agent binds RSPO1 with a K_{D} of about 0.1 nM or less. In certain embodiments, a RSPO1-binding agent described herein binds at least one other RSPO. In certain embodiments, a RSPO1-binding agent described herein that binds at least one other RSPO, binds at least one other RSPO with a K_{D} of about 100 nM or less, about 20 nM or less, about 10 nM or less, about 1 nM or less or about 0.1 nM or less. For example, in some embodiments, a RSPO1-binding agent also binds RSPO2, RSPO3, and/or RSPO4 with a K_{D} of about 10 nM or less. In some embodiments, a RSPO1-binding agent binds human RSPO1 with a K_{D} of about 0.1 nM or less. In some embodiments, the RSPO-binding agent binds both human RSPO and mouse RSPO with a K_{D} of about 10 nM or less. In some embodiments, a RSPO1-binding agent binds both human RSPO1 and mouse RSPO1 with a K_{D} of about 1 nM or less. In some embodiments, a RSPO1-binding agent binds both human RSPO1 and mouse RSPO1 with a K_{D} of about 0.1 nM or less. In some embodiments, the dissociation constant of the binding agent (e.g., an antibody) to a RSPO1 protein is the dissociation constant determined using a RSPO1 fusion protein comprising at least a portion of the RSPO1 protein immobilized on a Biacore chip.

In certain embodiments, a RSPO1-binding agent (e.g., an antibody) binds to human RSPO1 with a half maximal effective concentration (EC₅₀) of about 1 82 M or less, about 100 nM or less, about 40 nM or less, about 20 nM or less, about 10 nM or less, about 1 nM or less, or about 0.1 nM or less. In certain embodiments, a RSPO1-binding agent also binds to human RSPO2, RSPO3, and/or RSPO4 with an EC₅₀ of about 40 nM or less, about 20 nM or less, about 10 nM or less, about 1 nM or less or about 0.1 nM or less.

In certain embodiments, the RSPO1-binding agent is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, bivalent, bispecific, or multispecific. In some embodiments, the antibody is conjugated to a cytotoxic moiety. In some embodiments, the antibody is isolated. In some embodiments, the antibody is substantially pure.

The RSPO1-binding agents (e.g., antibodies) of the present invention can be assayed for specific binding by any method known in the art. The immunoassays that can be used include, but are not limited to, competitive and non-competitive assay systems using techniques such as Biacore analyses, FACS analyses, immunofluorescence, immunocytochemistry, Western blot analyses, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

For example, the specific binding of an antibody to human RSPO1 may be determined using ELISA. An ELISA assay comprises preparing antigen, coating wells of a 96 well microtiter plate with antigen, adding an anti-RSPO1 antibody conjugated to a detectable compound such as an enzymatic substrate (e.g. horseradish peroxidase or alkaline phosphatase) to the well, incubating for a period of time and detecting the presence of the antibody bound to the antigen. In some embodiments, the anti-RSPO1 antibody is not conjugated to a detectable compound, but instead a second conjugated antibody that recognizes the anti-RSPO1 antibody is added to the well. In some embodiments, instead of coating the well with the antigen, the anti-RSPO1 antibody can be coated to the well and a secondary antibody conjugated to a detectable compound can be added following the addition of the antigen to the coated well. One of skill in the art would be knowledgeable as to the parameters that can be modified to increase the signal detected as well as other variations of ELISAs known in the art.

In another example, the specific binding of an antibody to human RSPO1 may be determined using FACS. A FACS screening assay may comprise generating a cDNA construct that expresses an antigen as a fusion protein (e.g., RSPO1-Fc or RSPO1-CD4TM), transfecting the construct into cells, expressing the antigen on the surface of the cells, mixing an anti-RSPO1 antibody with the transfected cells, and incubating for a period of time. The cells bound by the anti-RSPO1 antibody may be identified by using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of a binding-agent (e.g., an anti-RSPO1 antibody) to an antigen (e.g., a RSPO1 protein) and the off-rate of a binding agent-antigen interaction can be determined by competitive binding assays. One example of a competitive binding assay is a radioimmunoassay comprising the incubation of labeled antigen (e.g., RSPO1-labeled with ³H or ¹²⁵H), or fragment or variant thereof, with a binding agent of interest in the presence of increasing amounts of unlabeled antigen followed by the detection of the agent bound to the labeled antigen. The affinity of the agent for an antigen (e.g., a RSPO1 protein) and the binding off-rates can be determined from the data by Scatchard plot analysis. In some embodiments, Biacore kinetic analysis is used to determine the binding on and off rates of antibodies that bind an antigen (e.g., a RSPO1 protein). Biacore kinetic analysis comprises analyzing the binding and dissociation of antibodies from chips with immobilized antigen (e.g., a RSPO1 protein) on their surface.

In certain embodiments, the invention provides a RSPO1-binding agent that specifically binds human RSPO1, wherein the RSPO1-binding agent comprises one, two, three, four, five, and/or six of the CDRs of antibody 89M5 (see Table 1). In some embodiments, the RSPO1-binding agent comprises one or more of the CDRs of 89M5, two or more of the CDRs of 89M5, three or more of the CDRs of 89M5, four or more of the CDRs of 89M5, five or more of the CDRs of 89M5, or all six of the CDRs of 89M5.

TABLE 1

| | 89M5 |
|---|---|
| HC CDR1 | TGYTMH (SEQ ID NO: 6) |
| HC CDR2 | GINPNNGGTTYNQNFKG (SEQ ID NO: 7) |
| HC CDR3 | KEFSDGYYFFAY (SEQ ID NO: 8) |
| LC CDR1 | KASQDVIFAVA (SEQ ID NO: 9) |
| LC CDR2 | WASTRHT (SEQ ID NO: 10) |
| LC CDR3 | QQHYSTPW (SEQ ID NO: 11) |

In certain embodiments, the invention provides a RSPO1-binding agent (e.g., an antibody) that specifically binds human RSPO1, wherein the RSPO1-binding agent comprises a heavy chain CDR1 comprising TGYTMH (SEQ ID NO:6), a heavy chain CDR2 comprising GINPNNGGTTYNQNFKG (SEQ ID NO:7), and a heavy chain CDR3 comprising KEFSDGYYFFAY (SEQ ID NO:8). In some embodiments, the RSPO1-binding agent further comprises a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11). In some embodiments, the RSPO1-binding agent comprises a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID

NO:11). In certain embodiments, the RSPO1-binding agent comprises: (a) a heavy chain CDR1 comprising TGYTMH (SEQ ID NO:6), a heavy chain CDR2 comprising GINPNNGGTTYNQNFKG (SEQ ID NO:7), and a heavy chain CDR3 comprising KEFSDGYYFFAY (SEQ ID NO:8), and (b) a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11).

In some embodiments, the RSPO1-binding agent is a modified version or variant of the 89M5 antibody. The hybridoma cell line producing the parental 89M5 antibody was deposited with American Type Culture Collection (ATCC), 10801 University Boulevard, Manassas, VA, USA, under the conditions of the Budapest Treaty on Jun. 30, 2011 and assigned ATCC deposit designation number PTA-11970. In some embodiments, the RSPO1-binding agents are antibody variants with desirable and/or improved properties from a production and/or purification perspective. In some embodiments, the RSPO1-binding agents are antibody variants with desirable properties such as improved solubility or improved stability. In some embodiments, the RSPO1-binding agents are antibody variants with desirable and/or improved properties from a therapeutic perspective. In some embodiments, the RSPO1-binding agent that has been modified or altered is a humanized version of 89M5. In some embodiments, the

RSPO1-binding agent that has been modified or altered is a humanized version of 89M5 referred to as h89M5-H8L5. Plasmids encoding the heavy chain and light chain of antibody h89M5-H8L5 were deposited with ATCC, 10801 University Boulevard, Manassas, Va., USA, under the conditions of the Budapest Treaty on Aug. 15, 2014 and assigned ATCC deposit designation numbers PTA-121494 and PTA-121495.

In certain embodiments, a RSPO1-binding agent is a humanized version of the 89M5 antibody. In some embodiments, the RSPO1-binding agent is a humanized version of the 89M5 antibody that has been modified or altered to have improved solubility. In some embodiments, the RSPO1-binding agent is a humanized version of the 89M5 antibody that has been modified or altered within the framework regions of the heavy chain variable region to have improved solubility. In some embodiments, the RSPO1-binding agent is a humanized version of the 89M5 antibody that has been modified or altered within the framework regions of the light chain variable region to have improved solubility. In some embodiments, the RSPO1-binding agent is a humanized version of the 89M5 antibody that has been modified or altered within the heavy chain to have improved solubility. In some embodiments, the RSPO1-binding agent is a humanized version of the 89M5 antibody that has been modified or altered within the light chain to have improved solubility. In some embodiments, the RSPO1-binding agent that has been modified or altered to have improved solubility is antibody h89M5-H8L5.

In some embodiments, the RSPO1-binding agent is a humanized version of the 89M5 antibody that has been modified or altered to have an improved antibody profile as assessed by size exclusion chromatography (SEC). In some embodiments, the RSPO1-binding agent is a humanized version of the 89M5 antibody that has been modified or altered within the framework regions of the heavy chain variable region to have an improved antibody profile as assessed by SEC. In some embodiments, the RSPO1-binding agent is a humanized version of the 89M5 antibody that has been modified or altered within the framework regions of the light chain variable region to have an improved antibody profile as assessed by SEC. In some embodiments, the RSPO1-binding agent is a humanized version of the 89M5 antibody that has been modified or altered within the heavy chain to have an improved antibody profile as assessed by SEC. In some embodiments, the RSPO1-binding agent is a humanized version of the 89M5 antibody that has been modified or altered within the light chain to have an improved antibody profile as assessed by SEC. In some embodiments, the RSPO1-binding agent that has been modified or altered to have an improved antibody profile as assessed by SEC is antibody h89M5-H8L5.

In certain embodiments, a RSPO1-binding agent comprises the heavy chain variable region and light chain variable region of antibody h89M5-H8L5. In certain embodiments, a RSPO1-binding agent comprises the heavy chain and light chain of antibody h89M5-H8L5 (with or without the leader sequence). In certain embodiments, a RSPO1-binding agent is antibody h89M5-H8L5.

In certain embodiments, the invention provides a RSPO1-binding agent (e.g., an antibody) that specifically binds RSPO1, wherein the RSPO1-binding agent comprises a heavy chain variable region having at least about 90% sequence identity to SEQ ID NO:32, and/or a light chain variable region having at least 90% sequence identity to SEQ ID NO:33. In certain embodiments, the RSPO1-binding agent comprises a heavy chain variable region having at least about 91%, 92%, 93%, 94%, 95%, 96%, 97%, 98% or 99% sequence identity to SEQ ID NO:32. In certain embodiments, the RSPO1-binding agent comprises a light chain variable region having at least about 91%, 92%, 93%, 94%, 95%, 96%, 97%, 98% or 99% sequence identity to SEQ ID NO:33. In certain embodiments, the RSPO1-binding agent comprises a heavy chain variable region having at least about 95% sequence identity to SEQ ID NO:32, and/or a light chain variable region having at least about 95% sequence identity to SEQ ID NO:33. In certain embodiments, the RSPO1-binding agent comprises a heavy chain variable region comprising SEQ ID NO:32 and/or a light chain variable region comprising SEQ ID NO:33. In certain embodiments, the RSPO1-binding agent comprises a heavy chain variable region consisting essentially of SEQ ID NO:32 and a light chain variable region consisting essentially of SEQ ID NO:33. In certain embodiments, the RSPO1-binding agent comprises a heavy chain variable region of SEQ ID NO:32 and a light chain variable region of SEQ ID NO:33.

In some embodiments, the RSPO1-binding agent is an antibody that comprises a heavy chain variable region having at least 90% sequence identity to SEQ ID NO:32 and a light chain variable region having at least 90% sequence identity to SEQ ID NO:33, wherein the antibody comprises a heavy chain CDR1 comprising TGYTMH (SEQ ID NO:6), a heavy chain CDR2 comprising GINPNNGGTTYNQNFKG (SEQ ID NO:7), and a heavy chain CDR3 comprising KEFSDGYYFFAY (SEQ ID NO:8), a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11).

In certain embodiments, the invention provides a RSPO1-binding agent (e.g., an antibody) that specifically binds RSPO1, wherein the RSPO1-binding agent comprises a heavy chain having at least 90% sequence identity to SEQ ID NO:35 and/or a light chain having at least 90% sequence identity to SEQ ID NO:37. In certain embodiments, the RSPO1-binding agent comprises a heavy chain having at least about 91%, 92%, 93%, 94%, 95%, 96%, 97%, 98% or 99% sequence identity to SEQ ID NO:35. In certain embodiments, the RSPO1-binding agent comprises a light chain having at least about 91%, 92%, 93%, 94%, 95%, 96%, 97%, 98% or 99% sequence identity to SEQ ID NO:37. In some embodiments, the RSPO1-binding agent comprises a heavy chain having at least 95% sequence identity to SEQ ID NO:35 and/or a light chain having at least 95% sequence identity to SEQ ID NO:37. In some embodiments, the RSPO1-binding agent comprises a heavy chain comprising SEQ ID NO:35 and/or a light chain comprising SEQ ID NO:37. In some embodiments, the RSPO1-binding agent comprises a heavy chain consisting essentially of SEQ ID NO:35, and a light chain consisting essentially of SEQ ID NO:37. In some embodiments, the RSPO1-binding agent comprises a heavy chain of SEQ ID NO:35, and a light chain of SEQ ID NO:37.

In some embodiments, the RSPO1-binding agent is an antibody that comprises a heavy chain having at least 90% sequence identity to SEQ ID NO:35 and a light chain having at least 90% sequence identity to SEQ ID NO:37, wherein the antibody comprises a heavy chain CDR1 comprising TGYTMH (SEQ ID NO:6), a heavy chain CDR2 comprising GINPNNGGTTYNQNFKG (SEQ ID NO:7), and a heavy chain CDR3 comprising KEFSDGYYFFAY (SEQ ID NO:8), a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11).

The invention provides polypeptides, including, but not limited to, antibodies that specifically bind human RSPO proteins. In some embodiments, the polypeptides bind human RSPO1.

In certain embodiments, the polypeptide comprises one, two, three, four, five, and/or six of the CDRs of antibody 89M5 (see Table 1 herein). In some embodiments, the polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the invention provides a polypeptide that specifically binds human RSPO1, wherein the polypeptide comprises an amino acid sequence having at least about 90% sequence identity to SEQ ID NO:32, and/or an amino acid sequence having at least about 90% sequence identity to

SEQ ID NO:33. In certain embodiments, the polypeptide comprises an amino acid sequence having at least about 95%, at least about 97%, or at least about 99% sequence identity to SEQ ID NO:32. In certain embodiments, the polypeptide comprises an amino acid sequence having at least about 95%, at least about 97%, or at least about 99% sequence identity to SEQ ID NO:33. In certain embodiments, the polypeptide comprises an amino acid sequence having at least about 95% sequence identity to SEQ ID NO:32, and/or an amino acid sequence having at least about 95% sequence identity to SEQ ID NO:33. In certain embodiments, the polypeptide comprises an amino acid sequence comprising SEQ ID NO:32 and/or an amino acid sequence comprising SEQ ID NO:33. In certain embodiments, the polypeptide comprises an amino acid sequence of SEQ ID NO:32 and/or an amino acid sequence of SEQ ID NO:33.

In some embodiments, the invention provides a polypeptide that specifically binds human

RSPO1, wherein the polypeptide comprises an amino acid sequence having at least about 90% sequence identity to SEQ ID NO:35, and/or an amino acid sequence having at least about 90% sequence identity to SEQ ID NO:37. In some embodiments, the polypeptide comprises an amino acid sequence having at least about 90% sequence identity to SEQ ID NO:34, and/or an amino acid sequence having at least about 90% sequence identity to SEQ ID NO:36. In certain embodiments, the polypeptide comprises an amino acid sequence having at least about 95%, at least about 97%, or at least about 99% sequence identity to SEQ ID NO:34. In certain embodiments, the polypeptide comprises an amino acid sequence having at least about 95%, at least about 97%, or at least about 99% sequence identity to SEQ ID NO:35. In certain embodiments, the polypeptide comprises an amino acid sequence having at least about 95%, at least about 97%, or at least about 99% sequence identity to SEQ ID NO:36. In certain embodiments, the polypeptide comprises an amino acid sequence having at least about 95%, at least about 97%, or at least about 99% sequence identity to SEQ ID NO:37. In certain embodiments, the polypeptide comprises an amino acid sequence having at least about 95% sequence identity to SEQ ID NO:35 and/or an amino acid sequence having at least about 95% sequence identity to SEQ ID NO:37. In certain embodiments, the polypeptide comprises an amino acid sequence having at least about 95% sequence identity to SEQ ID NO:34 and/or an amino acid sequence having at least about 95% sequence identity to SEQ ID NO:36. In certain embodiments, the polypeptide comprises an amino acid sequence comprising SEQ ID NO:35 and/or an amino acid sequence comprising SEQ ID NO:37. In certain embodiments, the polypeptide comprises an amino acid sequence comprising SEQ ID NO:34 and/or an amino acid sequence comprising SEQ ID NO:36. In certain embodiments, the polypeptide consists essentially of SEQ ID NO:35 and/or SEQ ID

NO:37. In certain embodiments, the polypeptide consists essentially of SEQ ID NO:34 and/or SEQ ID NO:36. In certain embodiments, the polypeptide is SEQ ID NO:35 and/or SEQ ID NO:37. In certain embodiments, the polypeptide is SEQ ID NO:34 and/or SEQ ID NO:36.

In some embodiments, a RSPO1-binding agent comprises a polypeptide comprising a sequence selected from the group consisting of: SEQ ID NO:32, SEQ ID NO:33, SEQ ID NO:34, SEQ ID NO:35, SEQ ID NO:36, and SEQ ID NO:37.

In certain embodiments, a RSPO1-binding agent comprises, consists essentially of, or consists of, the antibody h89M5-H8L5.

Many proteins, including antibodies, contain a signal sequence that directs the transport of the proteins to various locations. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell's outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or may be used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions as compared to a “native” or “parental” signal sequence. In some embodiments, the signal sequence of the polypeptide comprises amino acid substitutions and/or deletions that allow one cleavage site to be dominant, thereby resulting in a substantially homogeneous polypeptide with one N-terminus. In some embodiments, the signal sequence of the polypeptide is replaced with a different signal sequence. In some embodiments, a signal sequence of the polypeptide affects the expression level of the polypeptide. In some embodiments, a signal sequence of the polypeptide increases the expression level of the polypeptide. In some embodiments, a signal sequence of the polypeptide decreases the expression level of the polypeptide.

In certain embodiments, a RSPO1-binding agent (e.g., antibody) competes for specific binding to RSPO1 with an antibody that comprises a heavy chain variable region comprising SEQ ID NO:32 and a light chain variable region comprising SEQ ID NO:33. In certain embodiments, a RSPO1-binding agent competes for specific binding to RSPO1 with an antibody that comprises a heavy chain comprising SEQ ID NO:35 and a light chain comprising SEQ ID NO:37. In certain embodiments, a RSPO1-binding agent competes with antibody h89M5-H8L5 for specific binding to human RSPO1. In some embodiments, a RSPO1-binding agent or antibody competes for specific binding to RSPO1 in an in vitro competitive binding assay. In some embodiments, the RSPO1 is human RSPO1. In some embodiments, the RSPO1 is mouse RSPO1.

In certain embodiments, a RSPO1-binding agent (e.g., an antibody) binds the same epitope, or essentially the same epitope, on RSPO1 as an antibody of the invention. In another embodiment, a RSPO1-binding agent is an antibody that binds an epitope on RSPO1 that overlaps with the epitope on RSPO1 bound by an antibody of the invention. In certain embodiments, a RSPO1-binding agent (e.g., an antibody) binds the same epitope, or essentially the same epitope, on RSPO1 as antibody h89M5-H8L5. In another embodiment, the RSPO1-binding agent is an antibody that binds an epitope on RSPO1 that overlaps with the epitope on RSPO1 bound by antibody h89M5-H8L5.

In certain embodiments, the RSPO1-binding agent is an agent that competes for specific binding to RSPO1 with an antibody encoded by the plasmids having ATCC deposit designation number PTA-121494 and PTA-121495 (e.g., in a competitive binding assay).

In certain embodiments, a RSPO1-binding agent (e.g., an antibody) described herein binds human RSPO1 and modulates RSPO1 activity. In some embodiments, a RSPO1-binding agent is a RSPO1 antagonist and decreases RSPO1 activity. In some embodiments, a RSPO1-binding agent is a RSPO1 antagonist and decreases f3-catenin activity.

In certain embodiments, the RSPO1-binding agent (e.g., an antibody) is an antagonist of at least one human RSPO protein. In some embodiments, the RSPO1-binding agent is an antagonist of RSPO1 and inhibits RSPO1 activity. In certain embodiments, the RSPO1-binding agent inhibits RSPO1 activity by at least about 10%, at least about 20%, at least about 30%, at least about 50%, at least about 75%, at least about 90%, or about 100%. In some embodiments, the RSPO1-binding agent inhibits activity of one, two, three, or four RSPO proteins. In some embodiments, the RSPO1-binding agent inhibits activity of human RSPO1, RSPO2, RSPO3, and/or RSPO4. In certain embodiments, a RSPO1-binding agent that inhibits human RSPO1 activity is antibody h89M5-H8L5.

In certain embodiments, the RSPO1-binding agent inhibits RSPO1 signaling by at least about 10%, at least about 20%, at least about 30%, at least about 50%, at least about 75%, at least about 90%, or about 100%. In some embodiments, the RSPO1-binding agent inhibits signaling by one, two, three, or four RSPO proteins. In some embodiments, the RSPO1-binding agent inhibits signaling of human RSPO1, RSPO2, RSPO3, and/or RSPO4. In certain embodiments, a RSPO1-binding agent that inhibits RSPO1 signaling is antibody h89M5-H8L5.

In certain embodiments, the RSPO1-binding agent (e.g., antibody) is an antagonist of f3-catenin signaling. In certain embodiments, the RSPO1-binding agent inhibits 13-catenin signaling by at least about 10%, at least about 20%, at least about 30%, at least about 50%, at least about 75%, at least about 90%, or about 100%. In certain embodiments, a RSPO1-binding agent that inhibits f3-catenin signaling is antibody h89M5-H8L5.

In certain embodiments, the RSPO1-binding agent (e.g., antibody) inhibits binding of at least one

RSPO protein to a receptor. In certain embodiments, the RSPO1-binding agent inhibits binding of human RSPO1 to one or more of its receptors. In some embodiments, the RSPO1-binding agent inhibits binding of RSPO1 to at least one LGR protein. In some embodiments, the RSPO1-binding agent inhibits binding of RSPO1 to LGR4, LGRS, and/or LGR6. In some embodiments, a RSPO1-binding agent inhibits binding of RSPO1 to LGR4. In some embodiments, a RSPO1-binding agent inhibits binding of RSPO1 to

LGRS. In some embodiments, a RSPO1-binding agent inhibits binding of RSPO1 to LGR6. In certain embodiments, the inhibition of binding of a RSPO1-binding agent to at least one LGR protein is at least about 10%, at least about 25%, at least about 50%, at least about 75%, at least about 90%, or at least about 95%. In certain embodiments, a RSPO1-binding agent that inhibits binding of RSPO1 to at least one LGR protein further inhibits β-catenin signaling. In certain embodiments, a RSPO1-binding agent that inhibits binding of human RSPO1 to at least one LGR protein is antibody h89M5-H8L5.

In certain embodiments, the RSPO1-binding agent (e.g., antibody) blocks binding of at least one RSPO to a receptor. In certain embodiments, the RSPO1-binding agent blocks binding of human RSPO1 to one or more of its receptors. In some embodiments, the RSPO1-binding agent blocks binding of RSPO1 to at least one LGR protein. In some embodiments, the RSPO1-binding agent blocks binding of

RSPO1 to LGR4, LGRS, and/or LGR6. In some embodiments, a RSPO1-binding agent blocks binding of RSPO1 to LGR4. In some embodiments, a RSPO1-binding agent blocks binding of RSPO1 to LGRS. In some embodiments, a RSPO1-binding agent blocks binding of RSPO1 to LGR6. In certain embodiments, the blocking of binding of a RSPO1-binding agent to at least one LGR protein is at least about 10%, at least about 25%, at least about 50%, at least about 75%, at least about 90%, or at least about 95%. In certain embodiments, a RSPO1-binding agent that blocks binding of RSPO1 to at least one LGR protein further inhibits β-catenin signaling. In certain embodiments, a RSPO1-binding agent that blocks binding of human RSPO1 to at least one LGR protein is antibody h89M5-H8L5.

In certain embodiments, the RSPO1-binding agent (e.g., an antibody) inhibits β-catenin signaling. It is understood that a RSPO1-binding agent that inhibits β-catenin signaling may, in certain embodiments, inhibit signaling by one or more receptors in the β-catenin signaling pathway but not necessarily inhibit signaling by all receptors. In certain alternative embodiments, β-catenin signaling by all human receptors may be inhibited. In certain embodiments, β-catenin signaling by one or more receptors selected from the group consisting of LGR4, LGR5, and LGR6 is inhibited. In certain embodiments, the inhibition of β-catenin signaling by a RSPO1-binding agent is a reduction in the level of β-catenin signaling of at least about 10%, at least about 25%, at least about 50%, at least about 75%, at least about 90%, or at least about 95%. In some embodiments, a RSPO1-binding agent that inhibits β-catenin signaling is antibody h89M5-H8L5.

In certain embodiments, the RSPO1-binding agent (e.g., an antibody) inhibits activation of β-catenin. It is understood that a RSPO1-binding agent that inhibits activation of β-catenin may, in certain embodiments, inhibit activation of β-catenin by one or more receptors, but not necessarily inhibit activation of β-catenin by all receptors. In certain alternative embodiments, activation of β-catenin by all human receptors may be inhibited. In certain embodiments, activation of β-catenin by one or more receptors selected from the group consisting of LGR4, LGR5, and LGR6 is inhibited. In certain embodiments, the inhibition of activation of β-catenin by a RSPO1-binding agent is a reduction in the level of activation of β-catenin of at least about 10%, at least about 25%, at least about 50%, at least about 75%, at least about 90%, or at least about 95%. In some embodiments, a RSPO1-binding agent that inhibits activation of β-catenin is antibody h89M5-H8L5.

In vivo and in vitro assays for determining whether a RSPO1-binding agent (or candidate RSPO1-binding agent) inhibits β-catenin signaling are known in the art. For example, cell-based, luciferase reporter assays utilizing a TCF/Luc reporter vector containing multiple copies of the TCF-binding domain upstream of a firefly luciferase reporter gene may be used to measure β-catenin signaling levels in vitro (Gazit et al., 1999, Oncogene, 18; 5959-66; TOPflash, Millipore, Billerica Mass.). The level of β-catenin signaling in the presence of one or more Wnts (e.g., Wnt(s) expressed by transfected cells or provided by Wnt-conditioned media) with or without a RSPO protein or RSPO-conditioned media in the presence of a RSPO1-binding agent is compared to the level of signaling without the RSPO-binding agent present. In addition to the TCF/Luc reporter assay, the effect of a RSPO1-binding agent (or candidate agent) on β-catenin signaling may be measured in vitro or in vivo by measuring the effect of the agent on the level of expression of β-catenin-regulated genes, such as c-myc, cyclin D1, and/or fibronectin. In certain embodiments, the effect of a RSPO1-binding agent on β-catenin signaling may also be assessed by measuring the effect of the agent on the phosphorylation state of Dishevelled-1, Dishevelled-2, Dishevelled-3, LRPS, LRP6, and/or β-catenin.

In certain embodiments, the RSPO1-binding agents have one or more of the following effects: inhibit proliferation of tumor cells, inhibit tumor growth, reduce the tumorigenicity of a tumor, reduce the tumorigenicity of a tumor by reducing the frequency of cancer stem cells in the tumor, inhibit tumor growth, trigger cell death of tumor cells, induce cells in a tumor to differentiate, differentiate tumorigenic cells to a non-tumorigenic state, induce expression of differentiation markers in the tumor cells, prevent metastasis of tumor cells, or decrease survival of tumor cells.

In certain embodiments, the RSPO1-binding agents are capable of inhibiting tumor growth. In certain embodiments, the RSPO1-binding agents are capable of inhibiting tumor growth in vivo (e.g., in a xenograft mouse model, and/or in a human having cancer).

In certain embodiments, the RSPO1-binding agents are capable of reducing the tumorigenicity of a tumor. In certain embodiments, the RSPO1-binding agent or antibody is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication Number WO 2008/042236, U.S. Patent Publication No. 2008/0064049, and U.S. Patent Publication No. 2008/0178305.

In certain embodiments, the RSPO1-binding agents described herein have a circulating half-life in mice, cynomolgus monkeys, or humans of at least about 5 hours, at least about 10 hours, at least about 24 hours, at least about 3 days, at least about 1 week, or at least about 2 weeks. In certain embodiments, the RSPO1-binding agent is an IgG (e.g., IgG1 or IgG2) antibody that has a circulating half-life in mice, cynomolgus monkeys, or humans of at least about 5 hours, at least about 10 hours, at least about 24 hours, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0. Known methods of increasing the circulating half-life of antibody fragments lacking the Fc region include such techniques as PEGylation.

In some embodiments, the RSPO1-binding agents are polyclonal antibodies. Polyclonal antibodies can be prepared by any known method. In some embodiments, polyclonal antibodies are raised by immunizing an animal (e.g., rabbit, rat, mouse, goat, donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites, and the like, of the immunized animal. The polyclonal antibodies can be purified from serum or ascites according to standard methods in the art including, but not limited to, affinity chromatography, ion-exchange chromatography, gel electrophoresis, and dialysis.

In some embodiments, the RSPO1-binding agents are monoclonal antibodies. Monoclonal antibodies can be prepared using hybridoma methods known to one of skill in the art. In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof In some embodiments, the immunizing antigen can be a mouse protein or a portion thereof

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as Escherichia coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries expressing CDRs of the desired species.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the monoclonal antibody against human RSPO1 is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or binding capability using methods known to one skilled in the art. In some embodiments, the framework region residues of a human immunoglobulin are replaced with the corresponding residues in an antibody from a non-human species. In some embodiments, the humanized antibody can be further modified by the substitution of additional residues either in the framework region and/or within the replaced non-human residues to refine and optimize antibody specificity, affinity, and/or capability. In general, the humanized antibody will comprise substantially all of the CDRs that correspond to the non-human immunoglobulin whereas all, or substantially all, of the framework regions are those of a human immunoglobulin sequence. In some embodiments, the humanized antibody can also comprise at least a portion of an immunoglobulin constant region or domain (e.g., a Fc region), typically that of a human immunoglobulin. In certain embodiments, such humanized antibodies are used therapeutically because they may reduce antigenicity and HAMA (human anti-mouse antibody) responses when administered to a human subject.

In certain embodiments, the RSPO1-binding agent is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Techniques for the generation and use of antibody phage libraries are well-known by those of skill in the art. Affinity maturation strategies including, but not limited to, chain shuffling and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production.

This invention also encompasses bispecific antibodies that specifically recognize at least one human RSPO protein. Bispecific antibodies are capable of specifically recognizing and binding at least two different epitopes. The different epitopes can either be within the same molecule (e.g., two epitopes on human RSPO1) or on different molecules (e.g., one epitope on RSPO1 and one epitope on RSPO2). In some embodiments, the bispecific antibodies are monoclonal human or monoclonal humanized antibodies. In some embodiments, the antibodies can specifically recognize and bind a first antigen target, (e.g., RSPO1) as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA. In certain embodiments, the bispecific antibody specifically binds RSPO1, as well as an additional RSPO protein selected from the group consisting of RSPO2, RSPO3, and RSPO4.

Techniques for making bispecific antibodies are known by those skilled in the art. Bispecific antibodies can be intact antibodies or antibody fragments. Antibodies with more than two valencies are also contemplated. For example, trispecific antibodies can be prepared. Thus, in certain embodiments the antibodies to RSPO1 are multispecific.

In certain embodiments, the antibodies (or other polypeptides) described herein may be monospecific. For example, in certain embodiments, each of the one or more antigen-binding sites that an antibody contains is capable of binding (or binds) a homologous epitope on RSPO proteins. In certain embodiments, an antigen-binding site of a monospecific antibody described herein is capable of binding (or binds), for example, RSPO1 and RSPO2 (i.e., the same epitope is found on both RSPO1 and RSPO2 proteins).

In certain embodiments, the RSPO1-binding agent is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from Escherichia coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a RSPO protein or derivatives, fragments, analogs or homologs thereof In some embodiments, antibody fragments are linear antibody fragments. In certain embodiments, antibody fragments are monospecific or bispecific. In certain embodiments, the RSPO1-binding agent is a scFv. Various techniques can be used for the production of single-chain antibodies specific to one or more human RSPOs.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., a human RSPO1). In this regard, the variable region may comprise or be derived from any type of mammal that can be induced to mount a humoral response and generate immunoglobulins against the desired tumor associated antigen. As such, the variable region of the modified antibodies can be, for example, of human, murine, non-human primate (e.g. cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived from an antibody of different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

In certain embodiments, the variable domains in both the heavy and light chains are altered by replacement of one or more amino acid residues outside of the CDRs. In certain embodiments, the heavy and light chains are altered by replacement of one or more amino acid residues outside of the CDRs. In certain embodiments, during the humanization process a different heavy chain or light chain framework sequence is used while keeping the CDRs of the parental antibody intact.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ACH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the RSPO1-binding antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques well within the purview of the skilled artisan.

In certain embodiments, a RSPO1-binding agent that is an antibody does not have one or more effector functions. For instance, in some embodiments, the antibody has no ADCC activity, and/or no complement-dependent cytotoxicity (CDC) activity. In certain embodiments, the antibody does not bind an Fc receptor, and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art and described herein.

Thus, the present invention provides methods for producing an antibody that binds RSPO1. In some embodiments, the method for producing an antibody that binds RSPO1 comprises using hybridoma techniques. In some embodiments, a method for producing an antibody that binds human RSPO1 is provided. In some embodiments, the method comprises using amino acids 31-263 of human RSPO1. In some embodiments, the method comprises using amino acids 31-263 of SEQ ID NO:1. In some embodiments, the method of generating an antibody that binds at least one human RSPO protein comprises screening a human phage library. The present invention further provides methods of identifying an antibody that binds RSPO1. In some embodiments, the antibody is identified by screening by FACS for binding to RSPO1 or a portion thereof In some embodiments, the antibody is identified by screening using ELISA for binding to RSPO1. In some embodiments, the antibody is identified by screening by FACS for blocking of binding of RSPO1 to a human LGR protein. In some embodiments, the antibody is identified by screening for inhibition or blocking of β-catenin signaling.

In some embodiments, a method of generating an antibody to human RSPO1 protein comprises immunizing a mammal with a polypeptide comprising amino acids 31-263 of human RSPO1 (SEQ ID NO:1). In some embodiments, a method of generating an antibody to human RSPO1 protein comprises immunizing a mammal with a polypeptide comprising at least a portion of amino acids 21-263 of human RSPO1 (SEQ ID NO:1). In some embodiments, the method further comprises isolating antibodies or antibody-producing cells from the mammal In some embodiments, a method of generating a monoclonal antibody which binds RSPO1 protein comprises: (a) immunizing a mammal with a polypeptide comprising at least a portion of amino acids 21-263 of human RSPO1 (SEQ ID NO:1); (b) isolating antibody producing cells from the immunized mammal; (c) fusing the antibody-producing cells with cells of a myeloma cell line to form hybridoma cells. In some embodiments, the method further comprises (d) selecting a hybridoma cell expressing an antibody that binds RSPO1 protein. In some embodiments, the at least a portion of amino acids 21-263 of human RSPO1 is selected from the group consisting of SEQ ID NOs:2-5. In some embodiments, the at least a portion of amino acids 21-263 of human RSPO1 is SEQ ID NO:5. In some embodiments, the at least a portion of amino acids 21-263 of human RSPO1 is SEQ ID NO:2 or SEQ ID NO:3. In certain embodiments, the mammal is a mouse. In some embodiments, the antibody is selected using a polypeptide comprising at least a portion of amino acid 21-263 of human RSPO1 (SEQ ID NO:1). In certain embodiments, the polypeptide used for selection comprising at least a portion of amino acids 21-263 of human RSPO1 is selected from the group consisting of SEQ ID NOs:2-5. In some embodiments, the antibody binds RSPO1 and at least one other RSPO protein. In certain embodiments, the at least one other RSPO protein is selected from the group consisting of RSPO2, RSPO3 and RSPO4. In certain embodiments, the antibody binds RSPO1 and RSPO2. In certain embodiments, the antibody binds RSPO1 and RSPO3. In certain embodiments, the antibody binds RSPO1 and RSPO4. In certain embodiments, the antibody binds RSPO1, RSPO2, and RSPO3. In certain embodiments, the antibody binds RSPO1, RSPO2, and RSPO4. In certain embodiments, the antibody binds RSPO1, RSPO3, and RSPO4. In some embodiments, the antibody binds both human RSPO1 and mouse RSPO1.

In some embodiments, the antibody generated by the methods described herein is a RSPO1 antagonist. In some embodiments, the antibody generated by the methods described herein inhibits β-catenin signaling.

In some embodiments, a method of producing an antibody to at least one human RSPO protein comprises identifying an antibody using a membrane-bound heterodimeric molecule comprising a single antigen-binding site. In some non-limiting embodiments, the antibody is identified using methods and polypeptides described in International Publication WO 2011/100566.

In some embodiments, a method of producing an antibody to at least one human RSPO protein comprises screening an antibody-expressing library for antibodies that bind a human RSPO protein. In some embodiments, the antibody-expressing library is a phage library. In some embodiments, the screening comprises panning In some embodiments, the antibody-expressing library (e.g., phage library) is screened using at least a portion of amino acids 21-263 of human RSPO1 (SEQ ID NO:1). In some embodiments, antibodies identified in the first screening, are screened again using a different RSPO protein thereby identifying an antibody that binds RSPO1 and a second RSPO protein. In certain embodiments, the polypeptide used for screening comprises at least a portion of amino acids 21-263 of human RSPO1 selected from the group consisting of SEQ ID NOs:2-5. In some embodiments, the antibody identified in the screening binds RSPO1 and at least one other RSPO protein. In certain embodiments, the at least one other RSPO protein is selected from the group consisting of RSPO2, RSPO3 and RSPO4. In certain embodiments, the antibody identified in the screening binds RSPO1 and RSPO2. In certain embodiments, the antibody identified in the screening binds RSPO1 and RSPO3. In certain embodiments, the antibody identified in the screening binds RSPO1 and RSPO4. In some embodiments, the antibody identified in the screening binds both human RSPO1 and mouse RSPO1. In some embodiments, the antibody identified in the screening is a RSPO1 antagonist. In some embodiments, the antibody identified in the screening inhibits β-catenin signaling induced by RSPO1.

In certain embodiments, the antibodies described herein are isolated. In certain embodiments, the antibodies described herein are substantially pure.

In some embodiments of the present invention, the RSPO1-binding agents are polypeptides. The polypeptides can be recombinant polypeptides, natural polypeptides, or synthetic polypeptides comprising an antibody, or fragment thereof, that bind human RSPO1. It will be recognized in the art that some amino acid sequences of the invention can be varied without significant effect of the structure or function of the protein. Thus, the invention further includes variations of the polypeptides which show substantial activity or which include regions of an antibody, or fragment thereof, against human RSPO1. In some embodiments, amino acid sequence variations of RSPO1-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate any undesirable side effects of the polypeptides and variants. An overview for chemical moieties can be found in Remington: The Science and Practice of Pharmacy, 22st Edition, 2012, Pharmaceutical Press, London.

The isolated polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies, or fragments thereof, against human RSPO1. For example, recombinant expression vectors can be replicable DNA constructs which have synthetic or cDNA-derived DNA fragments encoding a polypeptide chain of a RSPO1-binding agent, an anti-RSPO1 antibody, or fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from Escherichia coli, including pCR1, pBR322, pMB9 and their derivatives, and wider host range plasmids, such as M13 and other filamentous single-stranded DNA phages.

Suitable host cells for expression of a RSPO1-binding polypeptide or antibody (or a RSPO1 protein to use as an antigen) include prokaryotes, yeast cells, insect cells, or higher eukaryotic cells under the control of appropriate promoters. Prokaryotes include gram-negative or gram-positive organisms, for example Escherichia coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are described by Pouwels et al. (1985, Cloning Vectors: A Laboratory Manual, Elsevier, New York, N.Y.). Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823.

Various mammalian or insect cell culture systems are used to express recombinant polypeptides. Expression of recombinant proteins in mammalian cells can be preferred because such proteins are generally correctly folded, appropriately modified, and completely functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), and HEK-293 (human embryonic kidney-derived) cell lines and variants thereof Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art.

Thus, the present invention provides cells comprising the RSPO1-binding agents described herein. In some embodiments, the cells produce the RSPO1-binding agents described herein. In certain embodiments, the cells produce an antibody. In certain embodiments, the cells produce antibody h89M5-H8L5.

The proteins (e.g., an anti-RSPO1 antibody) produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a RSPO-binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

In certain embodiments, the RSPO1-binding agent is a polypeptide that is not an antibody. A variety of methods for identifying and producing non-antibody polypeptides that bind with high affinity to a protein target are known in the art. In certain embodiments, phage display technology may be used to produce and/or identify a RSPO1-binding polypeptide. In certain embodiments, the polypeptide comprises a protein scaffold of a type selected from the group consisting of protein A, protein G, a lipocalin, a fibronectin domain, an ankyrin consensus repeat domain, and thioredoxin.

In certain embodiments, the RSPO1-binding agents or antibodies can be used in any one of a number of conjugated (i.e. an immunoconjugate or radioconjugate) or non-conjugated forms. In certain embodiments, the antibodies can be used in a non-conjugated form to harness the subject's natural defense mechanisms including complement-dependent cytotoxicity and antibody dependent cellular toxicity to eliminate the malignant or cancer cells.

In some embodiments, the RSPO1-binding agent (e.g., an antibody or polypeptide) is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of an antibody and one or more small molecule toxins, such as a calicheamicin, maytansinoids, a trichothene, and CC1065, and the derivatives of these toxins that have toxin activity, can also be used. Conjugates of an antibody and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. Polynucleotides

In certain embodiments, the invention encompasses polynucleotides comprising polynucleotides that encode a polypeptide that specifically binds at least one human RSPO or a fragment of such a polypeptide. The term “polynucleotides that encode a polypeptide” encompasses a polynucleotide which includes only coding sequences for the polypeptide as well as a polynucleotide which includes additional coding and/or non-coding sequences. For example, the invention provides a polynucleotide comprising a polynucleotide sequence that encodes an antibody to a human RSPO protein or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, a polynucleotide comprises a nucleotide sequence encoding an antibody that specifically binds human RSPO1 described herein. In certain embodiments, the polynucleotide comprises a polynucleotide encoding a polypeptide comprising a sequence selected from the group consisting of SEQ ID NO:32, SEQ ID NO:33, SEQ ID NO:34, SEQ ID NO:35, SEQ ID NO:36, and SEQ ID NO:37. In some embodiments, the polynucleotide comprises a polynucleotide sequence selected from the group consisting of SEQ ID NO:38, SEQ ID NO:39, SEQ ID NO:40, and SEQ ID NO:41.

In some embodiments, a plasmid comprises a polynucleotide comprising SEQ ID NO:38. In some embodiments, a plasmid comprises a polynucleotide comprising polynucleotide sequence SEQ ID NO:39. In some embodiments, a plasmid comprises a polynucleotide comprising polynucleotide sequence SEQ ID NO:40. In some embodiments, a plasmid comprises a polynucleotide comprising polynucleotide sequence SEQ ID NO:41. In some embodiments, a plasmid comprises a polynucleotide that encodes an amino acid sequence comprising SEQ ID NO:32 and/or SEQ ID NO:33. In some embodiments, a plasmid comprises a polynucleotide that encodes an amino acid sequence comprising SEQ ID NO:34 and/or SEQ ID NO:36. In some embodiments, a plasmid comprises a polynucleotide that encodes an amino acid sequence comprising SEQ ID NO:35 and/or SEQ ID NO:37.

In certain embodiments, the polynucleotide comprises a polynucleotide having a nucleotide sequence at least 80% identical, at least 85% identical, at least 90% identical, at least 95% identical, and in some embodiments, at least 96%, 97%, 98% or 99% identical to a polynucleotide comprising a sequence selected from the group consisting of SEQ ID NO:38, SEQ ID NO:39, SEQ ID NO:40, and SEQ

ID NO:41. Also provided is a polynucleotide that comprises a polynucleotide that hybridizes to SEQ ID NO:38, SEQ ID NO:39, SEQ ID NO:40, or SEQ ID NO:41. In certain embodiments, the hybridization is under conditions of high stringency.

In some embodiments, an antibody is encoded by a polynucleotide comprising SEQ ID NO:38 and SEQ ID NO:39. In some embodiments, an antibody is encoded by a polynucleotide comprising SEQ

ID NO:40 and SEQ ID NO:41.

In some embodiments, an antibody comprises the heavy chain variable region encoded by the plasmid deposited with ATCC as PTA-121494. In some embodiments, an antibody comprises the heavy chain encoded by the plasmid deposited with ATCC as PTA-121494. In some embodiments, an antibody comprises the light chain variable region encoded by the plasmid deposited with ATCC as PTA-121495. In some embodiments, an antibody comprises the light chain encoded by the plasmid deposited with ATCC as PTA-121495. In some embodiments, an antibody comprises the heavy chain variable region encoded by the plasmid deposited with ATCC as PTA-121494 and the light chain variable region encoded by the plasmid deposited with ATCC as PTA-121495. In some embodiments, an antibody comprises the heavy chain encoded by the plasmid deposited with ATCC as PTA-121494 and the light chain encoded by the plasmid deposited with ATCC as PTA-121495.

In certain embodiments, the polynucleotides comprise the coding sequence for the mature polypeptide fused in the same reading frame to a polynucleotide which aids, for example, in expression and secretion of a polypeptide from a host cell (e.g., a leader sequence or signal sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments, the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, which can be used in conjunction with other affinity tags.

The present invention further relates to variants of the hereinabove described polynucleotides encoding, for example, fragments, analogs, and/or derivatives.

In certain embodiments, the present invention provides polynucleotides comprising polynucleotides having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a RSPO-binding agent (e.g., an antibody), or fragment thereof, described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, the polynucleotide variants contain alterations which produce silent substitutions, additions, or deletions, but do not alter the properties or activities of the encoded polypeptide. In some embodiments, nucleotide variants are produced by silent substitutions due to the degeneracy of the genetic code. In some embodiments, nucleotide variants comprise nucleotide sequences which result in expression differences (e.g., increased or decreased expression), even though the amino acid sequence is not changed. Polynucleotide variants can be produced for a variety of reasons, for example, to optimize codon expression for a particular host (i.e., change codons in the human mRNA to those preferred by a bacterial host such as Escherichia coli).

In certain embodiments, the polynucleotides are isolated. In certain embodiments, the polynucleotides are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

IV. Methods of use and pharmaceutical compositions

The RSPO1-binding agents (including polypeptides and antibodies) of the invention are useful in a variety of applications including, but not limited to, therapeutic treatment methods, such as the treatment of cancer. In certain embodiments, the agents are useful for inhibiting β-catenin signaling, inhibiting tumor growth, inducing differentiation, reducing tumor volume, reducing the frequency of cancer stem cells in a tumor, and/or reducing the tumorigenicity of a tumor. The methods of use may be in vitro, ex vivo, or in vivo methods. In certain embodiments, a RSPO1-binding agent is an antagonist of human RSPO1.

In certain embodiments, the RSPO1-binding agents are used in the treatment of a disease associated with activation of β-catenin, increased β-catenin signaling, and/or aberrant β-catenin signaling. In certain embodiments, the disease is a disease dependent upon β-catenin signaling. In certain embodiments, the disease is a disease dependent upon β-catenin activation. In certain embodiments, the RSPO1-binding agents are used in the treatment of disorders characterized by increased levels of stem cells and/or progenitor cells. In some embodiments, the methods comprise administering a therapeutically effective amount of a RSPO1-binding agent (e.g., antibody) to a subject. In some embodiments, the subject is human.

In certain embodiments, the methods described herein comprise administering to a subject a therapeutically effective amount of an anti-RSPO1 antibody which comprises a heavy chain CDR1 comprising TGYTMH (SEQ ID NO:6), a heavy chain CDR2 comprising GINPNNGGTTYNQNFKG

(SEQ ID NO:7), and a heavy chain CDR3 comprising KEFSDGYYFFAY (SEQ ID NO:8), and/or a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11). In some embodiments, the methods described herein comprise administering to a subject a therapeutically effective amount of an anti-RSPO1 antibody which comprises a heavy chain variable region comprising SEQ ID NO:32 and a light chain variable region comprising SEQ ID NO:33. In some embodiments, the methods described herein comprise administering to a subject a therapeutically effective amount of an anti-RSPO1 antibody which comprises a heavy chain comprising SEQ ID NO:35 and a light chain comprising SEQ ID NO:37.

The present invention provides methods for inhibiting growth of a tumor using the RSPO1-binding agents described herein. In certain embodiments, the method of inhibiting growth of a tumor comprises contacting a cell with a RSPO1-binding agent in vitro. For example, an immortalized cell line or a cancer cell line is cultured in medium to which is added a RSPO1-binding agent to inhibit tumor growth. In some embodiments, tumor cells are isolated from a patient sample such as, for example, a tissue biopsy, pleural effusion, or blood sample and cultured in medium to which is added a RSPO1-binding agent to inhibit tumor growth. In some embodiments, the RSPO1-binding agent is an anti-RSPO1 antibody. In some embodiments, the anti-RSPO1 antibody is antibody h89M5-H8L5.

In some embodiments, the method of inhibiting growth of a tumor comprises contacting the tumor or tumor cells with a RSPO1-binding agent in vivo. In certain embodiments, contacting a tumor or tumor cell with a RSPO1-binding agent is undertaken in an animal model. For example, a RSPO1-binding agent may be administered to immunocompromised mice (e.g. NOD/SCID mice) which have tumor xenografts. In some embodiments, cancer cells or cancer stem cells are isolated from a patient sample such as, for example, a tissue biopsy, pleural effusion, or blood sample and injected into immunocompromised mice that are then administered a RSPO1-binding agent to inhibit tumor cell growth. In some embodiments, a RSPO1-binding agent is administered to the animal. In some embodiments, the RSPO1-binding agent is administered at the same time or shortly after introduction of tumorigenic cells into the animal to prevent tumor growth (“preventative model”). In some embodiments, the RSPO1-binding agent is administered as a therapeutic after tumors have grown to a specified size (“therapeutic model”). In some embodiments, the RSPO1-binding agent is an anti-RSPO1 antibody. In some embodiments, the anti-RSPO1 antibody is antibody h89M5-H8L5.

In certain embodiments, the method of inhibiting growth of a tumor comprises administering to a subject a therapeutically effective amount of a RSPO1-binding agent. In certain embodiments, the subject is a human. In certain embodiments, the subject has a tumor or has had a tumor which was removed. In some embodiments, the subject has a tumor with an elevated expression level of at least one RSPO protein (e.g., RSPO1, RSPO2, or RSPO3). In some embodiments, the RSPO1-binding agent is an anti-RSPO1 antibody. In some embodiments, the anti-RSPO1 antibody is antibody h89M5-H8L5.

In certain embodiments, the tumor is a tumor in which β-catenin signaling is active. In some embodiments, the tumor is a tumor in which β-catenin signaling is aberrant. In certain embodiments, the tumor comprises an inactivating mutation (e.g., a truncating mutation) in the APC tumor suppressor gene. In certain embodiments, the tumor does not comprise an inactivating mutation in the APC tumor suppressor gene. In some embodiments, the tumor comprises a wild-type APC gene. In some embodiments, the tumor does not comprise an activating mutation in the β-catenin gene. In certain embodiments, a cancer for which a subject is being treated involves such a tumor.

In certain embodiments, the tumor expresses RSPO1 to which a RSPO1-binding agent or antibody binds. In certain embodiments, the tumor has an elevated expression level of RSPO1 or over-expresses RSPO1. In some embodiments, the tumor has a high expression level of RSPO1. In general, the phrase “a tumor has an elevated expression level of” a protein (or similar phrases) refers to an expression level of a protein in a tumor as compared to a predetermined expression level of the protein. In some embodiments, the predetermined expression level is the expression level of the protein in normal tissue of the same tissue type. However, in some embodiments, the predetermined expression level of a protein is the average expression level of the protein within a group of tissue types. In some embodiments, the predetermined expression level is the expression level of the protein in other tumors of the same tissue type or a different tissue type. In certain embodiments, the tumor has elevated expression levels of RSPO2 or over-expresses RSPO2. In some embodiments, the tumor has a high expression level of RSPO2. In certain embodiments, the tumor has elevated expression levels of RSPO3 or over-expresses RSPO3. In some embodiments, the tumor has a high expression level of RSPO3. In certain embodiments, the tumor has elevated expression levels of RSPO4 or over-expresses RSPO4. In some embodiments, the tumor has a high expression level of RSPO4. In some embodiments, the tumor expresses elevated levels of RSPO1, RSPO2, RSPO3, and/or RSPO4 as compared to RSPO levels expressed in normal tissue. In some embodiments, the normal tissue is tissue of the same tissue type as the tumor.

In addition, the invention provides a method of inhibiting growth of a tumor in a subject, comprising administering a therapeutically effective amount of a RSPO1-binding agent to the subject. In certain embodiments, the tumor comprises cancer stem cells. In certain embodiments, the frequency of cancer stem cells in the tumor is reduced by administration of the RSPO1-binding agent. The invention also provides a method of reducing the frequency of cancer stem cells in a tumor, comprising contacting the tumor with an effective amount of a RSPO1-binding agent. In some embodiments, a method of reducing the frequency of cancer stem cells in a tumor in a subject, comprising administering to the subject a therapeutically effective amount of a RSPO1-binding agent (e.g., an anti-RSPO1 antibody) is provided. In some embodiments, the RSPO1-binding agent is an anti-RSPO1 antibody. In some embodiments, the anti-RSPO1 antibody is antibody h89M5-H8L5.

In some embodiments, the tumor is a solid tumor. In certain embodiments, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a lung tumor. In certain embodiments, the tumor is a pancreatic tumor. In some embodiments, the tumor is a colorectal tumor that comprises an inactivating mutation in the APC gene. In some embodiments, the tumor is a colorectal tumor that does not comprise an inactivating mutation in the APC gene. In some embodiments, the tumor is an ovarian tumor with an elevated expression level of RSPO1. In some embodiments, the tumor is a pancreatic tumor with an elevated expression level of RSPO2. In some embodiments, the tumor is a colon tumor with an elevated expression level of RSPO2. In some embodiments, the tumor is a lung tumor with an elevated expression level of RSPO2. In some embodiments, the tumor is a melanoma tumor with an elevated expression level of RSPO2. In some embodiments, the tumor is a breast tumor with an elevated expression level of RSPO2. In some embodiments, the tumor is a lung tumor with an elevated expression level of RSPO3. In some embodiments, the tumor is an ovarian tumor with an elevated expression level of RSPO3. In some embodiments, the tumor is a breast tumor with an elevated expression level of RSPO3. In some embodiments, the tumor is a colon tumor with an elevated expression level of RSPO3. In some embodiments, the tumor is a breast tumor with an elevated expression level of RSPO4. In some embodiments, the tumor is a lung tumor with an elevated expression level of RSPO4. In some embodiments, the tumor is an ovarian tumor with an elevated expression level of RSPO4. In some embodiments, the tumor is an ovarian tumor with a high expression level of RSPO1. In some embodiments, the tumor is a pancreatic tumor with a high expression level of RSPO2. In some embodiments, the tumor is a colon tumor with a high expression level of RSPO2. In some embodiments, the tumor is a lung tumor with a high expression level of RSPO2. In some embodiments, the tumor is a melanoma tumor with a high expression level of RSPO2. In some embodiments, the tumor is a breast tumor with a high expression level of RSPO2. In some embodiments, the tumor is a lung tumor with a high expression level of RSPO3. In some embodiments, the tumor is an ovarian tumor with a high expression level of RSPO3. In some embodiments, the tumor is a breast tumor with a high expression level of RSPO3. In some embodiments, the tumor is a colon tumor with a high expression level of RSPO3. In some embodiments, the tumor is a breast tumor with a high expression level of RSPO4. In some embodiments, the tumor is a lung tumor with a high expression level of RSPO4. In some embodiments, the tumor is an ovarian tumor with a high expression level of RSPO4.

The present invention further provides methods for treating cancer comprising administering a therapeutically effective amount of a RSPO1-binding agent to a subject. In certain embodiments, the cancer is characterized by cells expressing elevated levels of at least one RSPO protein as compared a predetermined level of the protein. In some embodiments, the predetermined expression level is the expression level of the same RSPO protein in normal tissue. In certain embodiments, the cancer is characterized by cells over-expressing RSPO1. In certain embodiments, the cancer is characterized by cells over-expressing RSPO2. In certain embodiments, the cancer is characterized by cells over-expressing RSPO3. In certain embodiments, the cancer over-expresses at least one RSPO protein selected from the group consisting of RSPO1, RSPO2, RSPO3, and/or RSPO4. In certain embodiments, the cancer is characterized by cells expressing β-catenin, wherein the RSPO1-binding agent interferes with RSPO-induced β-catenin signaling and/or activation. In some embodiments, the RSPO1-binding agent binds RSPO1, and inhibits or reduces growth of the cancer. In some embodiments, the RSPO1-binding agent binds RSPO1, interferes with RSPO1/LGR interactions, and inhibits or reduces growth of the cancer. In some embodiments, the RSPO1-binding agent binds RSPO1, inhibits β-catenin activation, and inhibits or reduces growth of the cancer. In some embodiments, the RSPO1-binding agent binds RSPO1, and reduces the frequency of cancer stem cells in the cancer. In some embodiments, the RSPO1-binding agent is an anti-RSPO1 antibody. In some embodiments, the anti-RSPO1 antibody is antibody h89M5-H8L5.

The present invention provides for methods of treating cancer comprising administering a therapeutically effective amount of a RSPO1-binding agent to a subject (e.g., a subject in need of treatment). In certain embodiments, the subject is a human. In certain embodiments, the subject has a cancerous tumor. In certain embodiments, the subject has had a tumor removed. In some embodiments, a method of treating cancer comprises administering a therapeutically effective amount of a RSPO1-binding agent to a subject, wherein the subject has a tumor that has elevated expression of at least one RSPO protein. In some embodiments, the subject has an ovarian tumor that has elevated expression of RSPO1 and is administered a RSPO1-binding agent. In some embodiments, the subject has an ovarian tumor that has elevated expression of RSPO1 and is administered an anti-RSPO1 antibody. In some embodiments, the subject has an ovarian tumor that has elevated expression of RSPO1 and is administered antibody h89M5-H8L5.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer.

In addition, the invention provides a method of reducing the tumorigenicity of a tumor in a subject, comprising administering to a subject a therapeutically effective amount of a RSPO1-binding agent. In certain embodiments, the tumor comprises cancer stem cells. In some embodiments, the tumorigenicity of a tumor is reduced by reducing the frequency of cancer stem cells in the tumor. In some embodiments, the methods comprise using the RSPO1-binding agents described herein. In certain embodiments, the frequency of cancer stem cells in the tumor is reduced by administration of a RSPO1-binding agent.

In certain embodiments, the methods further comprise a step of determining the expression level of at least one RSPO protein in the tumor or cancer. In some embodiments, the step of determining the expression level of RSPO in the tumor or cancer comprises determining the expression level of RSPO1,

RSPO2, RSPO3, and/or RSPO4. In some embodiments, the expression level of RSPO1, RSPO2, RSPO3, and/or RSPO4 in a tumor or cancer is compared to a predetermined expression level of RSPO1, RSPO2, RSPO3, and/or RSPO4. In some embodiments, the predetermined expression level is the expression level of RSPO1, RSPO2, RSPO3, and/or RSPO4 in normal tissue. In certain embodiments, the methods further comprise a step of determining if the tumor or cancer has an inactivating mutation in the APC gene. In some embodiments, the methods further comprise a step of determining if the tumor or cancer has an activating mutation in the 13-catenin gene. In some embodiments, determining the level of RSPO expression is done prior to treatment. In some embodiments, the subject is administered a RSPO1-binding agent or antibody describe herein if the tumor or cancer has an elevated level of RSPO expression as compared to a predetermined expression level of the same RSPO protein. In some embodiments, the subject is administered a RSPO-binding agent or antibody describe herein if the tumor or cancer has a mutation in the APC gene.

In addition, the present invention provides methods of identifying a human subject for treatment with a RSPO1-binding agent, comprising determining if the subject has a tumor that has an elevated level of RSPO expression as compared to a predetermined expression level of the same RSPO protein. In some embodiments, if the tumor has an elevated level of RSPO expression the subject is selected for treatment with an antibody that specifically binds RSPO1. In some embodiments, if selected for treatment, the subject is administered a RSPO1-binding agent or antibody describe herein. In certain embodiments, the subject has had a tumor removed. In some embodiments, the RSPO1-binding agent is an anti-RSPO1 antibody. In some embodiments, the anti-RSPO1 antibody is antibody h89M5-H8L5.

The present invention also provides methods of treating cancer in a human subject, comprising: (a) selecting a subject for treatment based, at least in part, on the subject having a cancer that has an elevated or high expression level of RSPO1, and (b) administering to the subject a therapeutically effective amount of a RSPO1-binding agent described herein. In some embodiments, the RSPO1-binding agent is an anti-RSPO1 antibody. In some embodiments, the anti-RSPO1 antibody is antibody h89M5-H8L5.

Methods for determining the level of RSPO expression in a cell, tumor or cancer are known by those of skill in the art. These methods include, but are not limited to, PCR-based assays, microarray analyses and nucleotide sequencing (e.g., NextGen sequencing) for nucleic acid expression. Other methods include, but are not limited, Western blot analyses, protein arrays, ELISAs, and FACS for protein expression.

Methods for determining whether a tumor or cancer has an elevated or high level of RSPO expression can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

Methods of treating a disease or disorder in a subject, wherein the disease or disorder is associated with aberrant (e.g., increased levels) β-catenin signaling are further provided. Methods of treating a disease or disorder in a subject, wherein the disease or disorder is characterized by an increased level of stem cells and/or progenitor cells are further provided. In some embodiments, the treatment methods comprise administering a therapeutically effective amount of a RSPO1-binding agent to the subject. In some embodiments, the RSPO1-binding agent is an anti-RSPO1 antibody. In some embodiments, the anti-RSPO1 antibody is antibody h89M5-H8L5.

The invention also provides a method of inhibiting β-catenin signaling in a cell comprising contacting the cell with an effective amount of a RSPO1-binding agent. In certain embodiments, the cell is a tumor cell. In certain embodiments, the method is an in vivo method wherein the step of contacting the cell with the RSPO1-binding agent comprises administering a therapeutically effective amount of the RSPO1-binding agent to the subject. In some embodiments, the method is an in vitro or ex vivo method.

In certain embodiments, the RSPO1-binding agent inhibits β-catenin signaling. In some embodiments, the RSPO1-binding agent inhibits activation of β-catenin. In certain embodiments, the RSPO1-binding agent interferes with a RSPO/LGR interaction. In certain embodiments, the LGR is LGR4, LGRS, and/or LGR6. In certain embodiments, the LGR is LGR4. In certain embodiments, the LGR is LGRS. In certain embodiments, the LGR is LGR6. In some embodiments, the RSPO1-binding agent is an anti-RSPO1 antibody. In some embodiments, the anti-RSPO1 antibody is antibody h89M5-H8L5.

The use of the RSPO1-binding agents described herein to induce the differentiation of cells, including, but not limited to tumor cells, is also provided. In some embodiments, methods of inducing cells to differentiate comprise contacting the cells with an effective amount of a RSPO1-binding agent described herein. In certain embodiments, methods of inducing cells in a tumor in a subject to differentiate comprise administering a therapeutically effective amount of a RSPO1-binding agent to the subject. In some embodiments, methods for inducing differentiation markers on tumor cells comprise administering a therapeutically effective amount of a RSPO1-binding agent. In some embodiments, the tumor is a solid tumor. In some embodiments, the tumor is selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is an ovarian tumor. In certain other embodiments, the tumor is a colon tumor. In some embodiments, the tumor is a lung tumor. In certain embodiments, the method is an in vivo method. In certain embodiments, the method is an in vitro method. In some embodiments, the RSPO1-binding agent is an anti-RSPO1 antibody. In some embodiments, the anti-RSPO1 antibody is antibody h89M5-H8L5.

The invention further provides methods of differentiating tumorigenic cells into non-tumorigenic cells comprising contacting the tumorigenic cells with a RSPO1-binding agent. In some embodiments, the method comprises administering the RSPO1-binding agent to a subject that has a tumor comprising tumorigenic cells or that has had such a tumor removed. In certain embodiments, the tumorigenic cells are ovarian tumor cells. In certain embodiments, the tumorigenic cells are colon tumor cells. In some embodiments, the tumorigenic cells are lung tumor cells. In some embodiments, the RSPO1-binding agent is an anti-RSPO1 antibody. In some embodiments, the anti-RSPO1 antibody is antibody h89M5-H8L5.

In certain embodiments, the disease treated with the RSPO1-binding agents described herein is not a cancer. For example, the disease may be a metabolic disorder such as obesity or diabetes (e.g., type II diabetes) (Jin T., 2008, Diabetologia, 51:1771-80). Alternatively, the disease may be a bone disorder such as osteoporosis, osteoarthritis, or rheumatoid arthritis (Corr M., 2008, Nat. Clin. Pract. Rheumatol., 4:550-6; Day et al., 2008, Bone Joint Surg. Am., 90 Suppl 1:19-24). The disease may also be a kidney disorder, such as a polycystic kidney disease (Harris et al., 2009, Ann. Rev. Med., 60:321-337; Schmidt-Ott et al., 2008, Kidney Int., 74:1004-8; Benzing et al., 2007, J. Am. Soc. Nephroi., 18:1389-98). Alternatively, eye disorders including, but not limited to, macular degeneration and familial exudative vitreoretinopathy may be treated (Lad et al., 2009, Stem Cells Dev., 18:7-16). Cardiovascular disorders, including myocardial infarction, atherosclerosis, and valve disorders, may also be treated (Al-Aly Z., 2008, Transl. Res., 151:233-9; Kobayashi et al., 2009, Nat. Cell Biol., 11:46-55; van Gijn et al., 2002, Cardiovasc. Res., 55:16-24; Christman et al., 2008, Am. J. Physiol. Heart Circ. Physiol., 294:H2864-70). In some embodiments, the disease is a pulmonary disorder such as idiopathic pulmonary arterial hypertension or pulmonary fibrosis (Laumanns et al., 2008, Am. J. Respir. Cell Mol. Biol., 2009, 40:683-691; Königshoff et al., 2008, PLoS ONE, 3:e2142). In some embodiments, the disease treated with the RSPO1-binding agent is a liver disease, such as cirrhosis or liver fibrosis (Cheng et al., 2008, Am. J. Physiol. Gastrointest. Liver Physiol., 294:G39-49).

The present invention provides compositions comprising the RSPO1-binding agents described herein. The present invention also provides pharmaceutical compositions comprising the RSPO1-binding agents described herein. In certain embodiments, the pharmaceutical compositions comprise a pharmaceutically acceptable vehicle. These pharmaceutical compositions find use in inhibiting tumor growth and treating cancer in a subject (e.g., a human patient).

Formulations are prepared for storage and use by combining a purified antibody or agent of the present invention with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose, or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The RSPO1-binding agents described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22st Edition, 2012, Pharmaceutical Press, London.

In certain embodiments, pharmaceutical formulations include a RSPO1-binding agent (e.g., an antibody) of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the RSPO1-binding agents described herein can be produced. Suitable examples of sustained-release preparations include semi-permeable matrices of solid hydrophobic polymers containing a RSPO-binding agent (e.g., an antibody), where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(-)-3-hydroxybutyric acid.

In certain embodiments, in addition to administering a RSPO1-binding agent (e.g., an antibody), a method or treatment further comprises administering at least one additional therapeutic agent. An additional therapeutic agent can be administered prior to, concurrently with, and/or subsequently to, administration of the RSPO1-binding agent. Pharmaceutical compositions comprising the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects (e.g., inhibits or kills) non-tumorigenic cells and a therapeutic agent that affects (e.g., inhibits or kills) tumorigenic CSCs.

In some embodiments, the combination of a RSPO1-binding agent and at least one additional therapeutic agent results in additive or synergistic results. In some embodiments, the combination therapy results in an increase in the therapeutic index of the RSPO1-binding agent. In some embodiments, the combination therapy results in an increase in the therapeutic index of the additional agent(s). In some embodiments, the combination therapy results in a decrease in the toxicity and/or side effects of the RSPO1-binding agent. In some embodiments, the combination therapy results in a decrease in the toxicity and/or side effects of the additional agent(s).

Useful classes of therapeutic agents that may be used in combination with a RSPO1-binding agent include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the additional therapeutic agent is an alkylating agent, an antimetabolite, an antimitotic, a topoisomerase inhibitor, or an angiogenesis inhibitor. In some embodiments, the additional therapeutic agent is a platinum complex such as carboplatin or cisplatin. In some embodiments, the additional therapeutic agent is a platinum complex in combination with a taxane.

Therapeutic agents that may be administered in combination with a RSPO1-binding agent include chemotherapeutic agents. Thus, in some embodiments, the method or treatment involves the administration of a RSPO1-binding agent or antibody of the present invention in combination with a chemotherapeutic agent or cocktail of multiple different chemotherapeutic agents. Treatment with a RSPO1-binding agent (e.g., an antibody) can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions, following standard-of-care procedures, or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa;

ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin. In certain embodiments, the additional therapeutic agent is paclitaxel. In some embodiments, a method comprises administering anti-RSPO1 antibody h89M5-H8L5 in combination with cisplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HC1, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan. Thus, in some embodiments, a method comprises administering a RSPO1-binding agent in combination with a topoisomerase inhibitor. In some embodiments, a method comprises administering anti-RSPO1 antibody h89M5-H8L5 in combination with irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine (XELODA) methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine. Thus, in some embodiments, a method comprises administering a RSPO1-binding agent in combination with an anti-metabolite. In some embodiments, a method comprises administering anti-RSPO1 antibody h89M5-H8L5 in combination with gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof In some embodiments, the antimitotic agent is an inhibitor of kinesin Eg5 or an inhibitor of a mitotic kinase such as Aurora A or Plk1. In certain embodiments, where the chemotherapeutic agent administered in combination with a RSPO-binding agent is an anti-mitotic agent, the cancer or tumor being treated is breast cancer or a breast tumor.

In some embodiments, an additional therapeutic agent comprises an agent such as a small molecule. For example, treatment can involve the combined administration of a RSPO1-binding agent (e.g. an antibody) of the present invention with a small molecule that acts as an inhibitor against additional tumor-associated antigens including, but not limited to, EGFR, ErbB2, HER2, and/or VEGF. In certain embodiments, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is a molecule that inhibits β-catenin signaling.

In some embodiments, an additional therapeutic agent comprises a biological molecule, such as an antibody. For example, treatment can involve the combined administration of a RSPO1-binding agent (e.g. an antibody) of the present invention with other antibodies against additional tumor-associated antigens including, but not limited to, antibodies that bind EGFR, ErbB2, HER2, and/or VEGF. In some embodiments, the additional therapeutic agent is a second anti-RSPO antibody. In some embodiments, the additional therapeutic agent is an anti-RSPO2 antibody, an anti-RSPO3 antibody, and/or an anti-RSPO4 antibody used in combination with an anti-RSPO1 antibody. In certain embodiments, the additional therapeutic agent is an antibody specific for an anti-cancer stem cell marker. In some embodiments, the additional therapeutic agent is an antibody that binds a component of the Notch pathway. In some embodiments, the additional therapeutic agent is an antibody that binds a component of the Wnt pathway. In certain embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits f3-catenin signaling. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), trastuzumab (HERCEPTIN), panitumumab (VECTIBIX), or cetuximab (ERBITUX).

In some embodiments, the methods described herein comprise administering a therapeutically effective amount of a RSPO1-binding agent in combination with Wnt pathway inhibitors. In some embodiments, the Wnt pathway inhibitors are frizzled (FZD) protein binding agents, “FZD-binding agents”. Non-limiting examples of FZD-binding agents can be found in U.S. Pat. No. 7,982,013. FZD-binding agents may include, but are not limited to, anti-FZD antibodies. In some embodiments, a method comprises administering a RSPO1-binding agent in combination with an anti-FZD antibody. In some embodiments, a method comprises administering a RSPO1-binding agent in combination with the anti-FZD antibody 18R5. In some embodiments, the Wnt pathway inhibitors are Wnt protein binding agents, “Wnt-binding agents”. Non-limiting examples of Wnt-binding agents can be found in U.S. Patent Nos. 7,723,477 and 7,947,277; and International Publications WO 2011/088127 and WO 2011/088123. Wnt-binding agents may include, but are not limited to, anti-Wnt antibodies and FZD-Fc soluble receptors. In some embodiments, a method comprises administering a RSPO1-binding agent in combination with a FZD-Fc soluble receptor. In some embodiments, a method comprises administering a RSPO1-binding agent in combination with a FZD8-Fc soluble receptor. In some embodiments, a method comprises administering a RSPO1-binding agent in combination with an anti-FZD antibody. In some embodiments, a method comprises administering anti-RSPO1 antibody h89M5-H8L5 in combination with an anti-FZD antibody. In some embodiments, a method comprises administering anti-RSPO1 antibody h89M5-H8L5 in combination with anti-FZD antibody 18R5. In some embodiments, a method comprises administering anti-RSPO1 antibody h89M5-H8L5 in combination with a FZD-Fc soluble receptor. In some embodiments, a method comprises administering anti-RSPO1 antibody h89M5-H8L5 in combination with a FZD8-Fc soluble receptor.

Furthermore, treatment with a RSPO1-binding agent described herein can include combination treatment with other biologic molecules, such as one or more cytokines (e.g., lymphokines, interleukins, tumor necrosis factors, and/or growth factors) or can be accompanied by surgical removal of tumors, cancer cells or any other therapy deemed necessary by a treating physician.

In certain embodiments, the treatment involves the administration of a RSPO1-binding agent (e.g. an antibody) of the present invention in combination with radiation therapy. Treatment with a RSPO-binding agent can occur prior to, concurrently with, or subsequent to administration of radiation therapy. Dosing schedules for such radiation therapy can be determined by the skilled medical practitioner.

It will be appreciated that the combination of a RSPO1-binding agent and at least one additional therapeutic agent may be administered in any order or concurrently. In some embodiments, the RSPO1-binding agent will be administered to patients that have previously undergone treatment with a second therapeutic agent. In certain other embodiments, the RSPO1-binding agent and a second therapeutic agent will be administered substantially simultaneously or concurrently. For example, a subject may be given a RSPO1-binding agent (e.g., an antibody) while undergoing a course of treatment with a second therapeutic agent (e.g., chemotherapy). In certain embodiments, a RSPO1-binding agent will be administered within 1 year of the treatment with a second therapeutic agent. In certain alternative embodiments, a RSPO1-binding agent will be administered within 10, 8, 6, 4, or 2 months of any treatment with a second therapeutic agent. In certain other embodiments, a RSPO1-binding agent will be administered within 4, 3, 2, or 1 weeks of any treatment with a second therapeutic agent. In some embodiments, a RSPO1-binding agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

In some embodiments, a treatment or dosing regimen may be limited to a specific number of administrations or “cycles”. A “cycle” may be a dosing schedule that is well-known or commonly used by those of skill in the art for a standard-of-care therapeutic agent. For example, a cycle of paclitaxel may be administration once a week for 3 weeks of a month cycle (there is one week of no administration each month). In some embodiments of the methods described herein, the RSPO1-binding agent is administered for 2, 3, 4, 5, 6, 7, 8, or more cycles. In some embodiments of the methods described herein, the additional therapeutic agent is administered for 2, 3, 4, 5, 6, 7, 8, or more cycles.

For the treatment of a disease, the appropriate dosage of an RSPO1-binding agent (e.g., an antibody) of the present invention depends on the type of disease to be treated, the severity and course of the disease, the responsiveness of the disease, whether the RSPO1-binding agent or antibody is administered for therapeutic or preventative purposes, previous therapy, the patient's clinical history, and so on, all at the discretion of the treating physician. The RSPO1-binding agent or antibody can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual antibody or agent. The administering physician can easily determine optimum dosages, dosing methodologies, and repetition rates. In certain embodiments, dosage is from 0.01 μg to 100mg/kg of body weight, from 0.1 μg to 100 mg/kg of body weight, from 1 μg to 100mg/kg of body weight, from 1 mg to 100 mg/kg of body weight, 1 mg to 80 mg/kg of body weight from 10 mg to 100 mg/kg of body weight, from 10 mg to 75 mg/kg of body weight, or from 10 mg to 50 mg/kg of body weight. In certain embodiments, the dosage of the antibody or other RSPO1-binding agent is from about 0.1 mg to about 20 mg/kg of body weight. In certain embodiments, dosage can be given once or more daily, weekly, monthly, or yearly. In certain embodiments, the antibody or other RSPO1-binding agent is given once every week, once every two weeks or once every three weeks.

In some embodiments, a RSPO1-binding agent (e.g., an antibody) may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

Thus, the present invention provides methods of treating cancer in a subject comprising using an intermittent dosing strategy for administering one or more agents, which may reduce side effects and/or toxicities associated with administration of a RSPO1-binding agent, chemotherapeutic agent, etc. In some embodiments, a method for treating cancer in a human subject comprises administering to the subject a therapeutically effective dose of a RSPO1-binding agent in combination with a therapeutically effective dose of a chemotherapeutic agent, wherein one or both of the agents are administered according to an intermittent dosing strategy. In some embodiments, the intermittent dosing strategy comprises administering an initial dose of a RSPO1-binding agent to the subject, and administering subsequent doses of the RSPO1-binding agent about once every 2 weeks. In some embodiments, the intermittent dosing strategy comprises administering an initial dose of a RSPO1-binding agent to the subject, and administering subsequent doses of the RSPO1-binding agent about once every 3 weeks. In some embodiments, the intermittent dosing strategy comprises administering an initial dose of a RSPO1-binding agent to the subject, and administering subsequent doses of the RSPO1-binding agent about once every 4 weeks. In some embodiments, the RSPO1-binding agent is administered using an intermittent dosing strategy and the chemotherapeutic agent is administered weekly.

### V. Kits Comprising RSPO1-Binding Agents

The present invention provides kits that comprise the RSPO1-binding agents (e.g., antibodies) described herein and that can be used to perform the methods described herein. In certain embodiments, a kit comprises at least one purified antibody against human RSPO1 in one or more containers. In some embodiments, the kits contain all of the components necessary and/or sufficient to perform a detection assay, including all controls, directions for performing assays, and any necessary software for analysis and presentation of results. One skilled in the art will readily recognize that the disclosed RSPO1-binding agents of the present invention can be readily incorporated into one of the established kit formats which are well known in the art.

Further provided are kits comprising a RSPO1-binding agent (e.g., an anti-RSPO1 antibody), as well as at least one additional therapeutic agent. In certain embodiments, the second (or more) therapeutic agent is a chemotherapeutic agent. In certain embodiments, the second (or more) therapeutic agent is a Wnt pathway inhibitor. In certain embodiments, the second (or more) therapeutic agent is an angiogenesis inhibitor.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Generation of Anti-RSPO1 Monoclonal Antibodies

The generation of anti-RSPO1 antibodies has been described (see, e.g., U.S. Pat. No. 8,802,097 and International Publication No. WO 2013/012747). The hybridoma cell line expressing anti-RSPO1 antibody 89M5 was deposited with the ATCC, 10801 University Boulevard, Manassas, Va., USA, under the conditions of the Budapest Treaty on June 30, 2011 and assigned ATCC deposit designation number PTA-11970. The heavy chain and light chain CDRs of 89M5 are listed in Table 1 herein. The amino acid sequences of the heavy chain and light chain variable regions of 89M5 are SEQ ID NO:12 and SEQ ID NO:13. The amino acid sequences of the heavy chain and light chain of 89M5 are SEQ ID NO:14 and SEQ ID NO:16; the nucleotide sequences of the heavy chain and light chain of 89M5 are SEQ ID NO:20 and SEQ ID NO:21 (with the predicted signal sequence).

A first generation humanized version of anti-RSPO1 antibody 89M5 was generated and named h89M5-H2L2. Subsequently, anti-RSPO1 antibody h89M5-H2L2 was observed to have an anomalous antibody profile when characterized by size exclusion chromatography (SEC). Furthermore, this antibody was observed to have low solubility when formulated for in vivo use than desired.

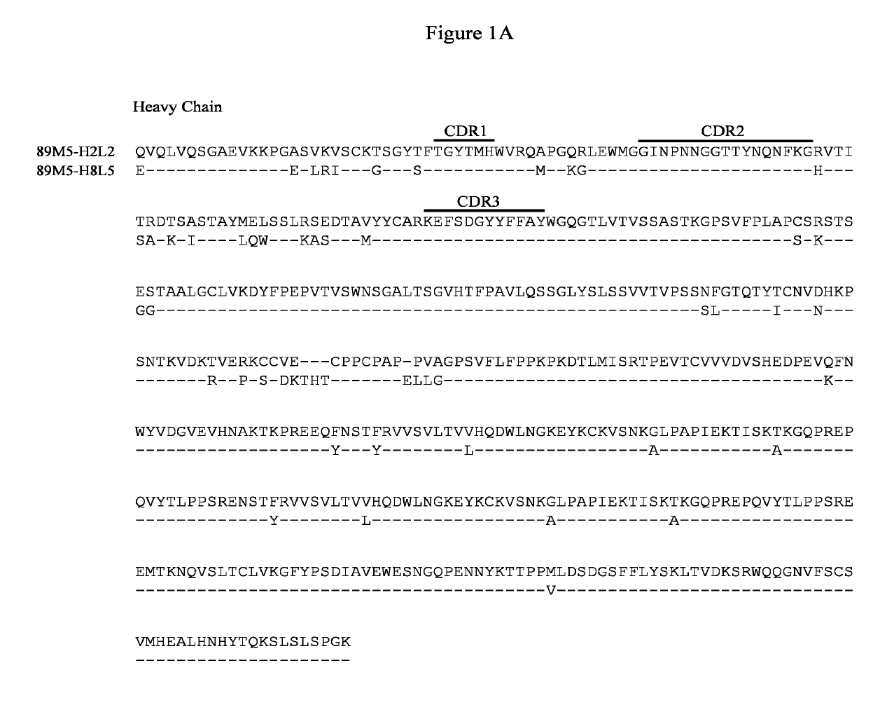

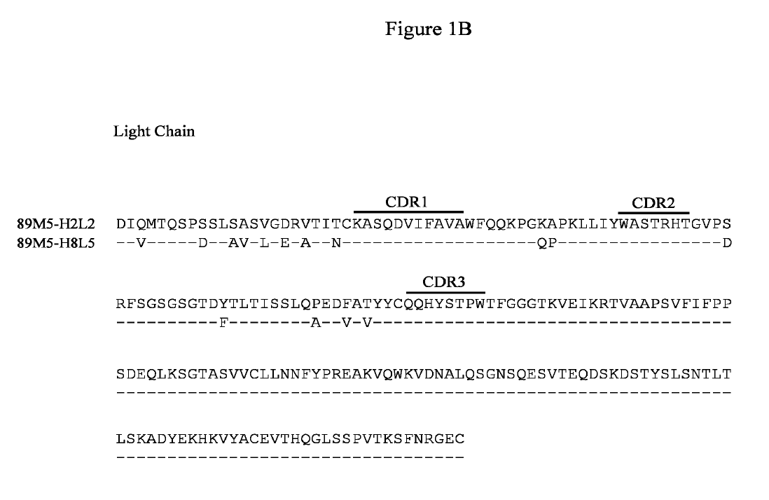

Therefore, additional second generation humanized versions of 89M5 were generated and characterized. Plasmids encoding the heavy chain and light chain of second generation humanized antibody h89M5-H8L5 were deposited with ATCC, 10801 University Boulevard, Manassas, Va., USA, under the conditions of the Budapest Treaty on August 15, 2014 and assigned ATCC deposit designation numbers PTA-121494 and PTA-121495. The amino acid sequence differences between the heavy chain and light chain of first generation humanized anti-RSPO1 antibody h89M5-H2L2 and second generation humanized anti-RSPO1 antibody h89M5-H8L5 are shown in FIGS. 1A and 1B. These antibodies differ in the sequence of their heavy chains (H2 and H8) and their light chains (L2 and L5) but retain the same heavy chain and light chain CDR amino acid sequences.

### Example 2

### FACS Binding of Anti-RSPO1 Antibodies

HEK-293 cells were transiently transfected with an expression vector encoding FLAG-RSPO1furin-CD4TM-GFP. FLAG-RSPO1furin-CD4TM-GFP is a chimeric fusion protein enabling cell surface expression of the N-terminal furin-like domains of human RSPO1. FLAG-RSPO1furin-CD4TM-GFP transfected cells were incubated in the presence of first generation humanized anti-RSPO1 antibody h89M5-H2L2 and second generation humanized anti-RSPO1 antibody h89M5-H8L5. Serial dilutions of each antibody were examined for their ability to bind to the RSPO1-expressing cells. The cells were stained with allphycocyanin (APC)-conjugated anti-IgG to detect cells bound by antibody. The cells were analyzed on a FACSCalibur instrument (BD Biosciences, San Jose, Calif.) and the data was processed using FlowJo software.

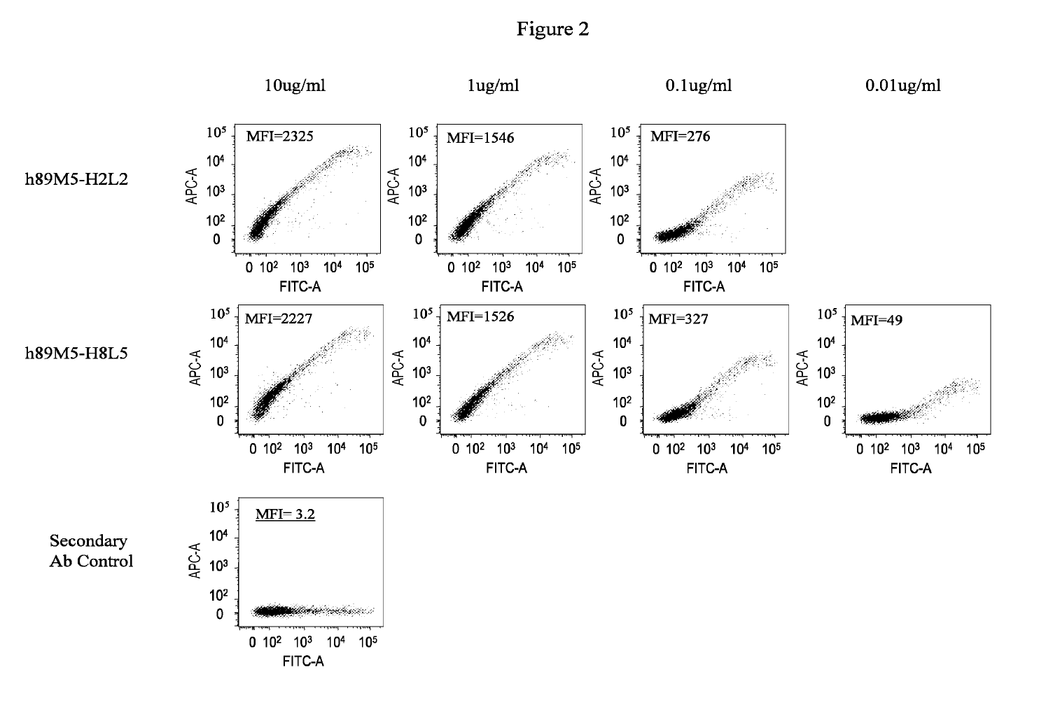

As shown in FIG. 2, this study indicated that the second generation humanized anti-RSPO1 antibody h89M5-H8L5 bound to human RSPO1 in a dose-dependent manner and with similar activity as the first generation anti-RSPO1 antibody h89M5-H2L2.

### Example 3

### Size Exclusion Chromatography of Anti-RSPO1 Antibodies

Size exclusion chromatography (SEC) analysis was performed on humanized versions of the anti-RSPO1 antibody OMP-89M5. Size exclusion chromatography can be used to assess purity of antibodies by distinguishing monomeric (i.e., intact) antibodies from smaller antibody fragments and/or larger antibody aggregates. The first generation humanized antibody h89M5-H2L2 was produced, purified, and formulated at a concentration of 20.9 mg/ml in 50 mM histidine, 2.5% PEG 400, pH 6.0. The second generation humanized antibody h89M5-H8L5 was expressed from a pool of stably transfected cells, purified by Protein A chromatography, and formulated at a concentration of 2.8 mg/ml in 0.1M glycine/Bis Tris, pH 5.0.

The samples h89M5-H2L2 and h89M5-H8L5 were analyzed using a Waters UPLC system (Acquity UPLC H-class Biosystem) with a dual wavelength UV detector. Samples were prepared at a concentration of 1 mg/ml in SEC buffer (100 mM sodium phosphate, 150 mM NaCl, pH 6.8). 10 μl of each sample was injected onto a Waters Acquity UPLC BEH200 SEC 1.7 μm 4.6×150mm column and run isocratically at 0.4 ml/min for 7 minutes. BioRad molecule weight (MW) markers were run as standards. Protein peaks were detected with the UV detector at 215 nm.

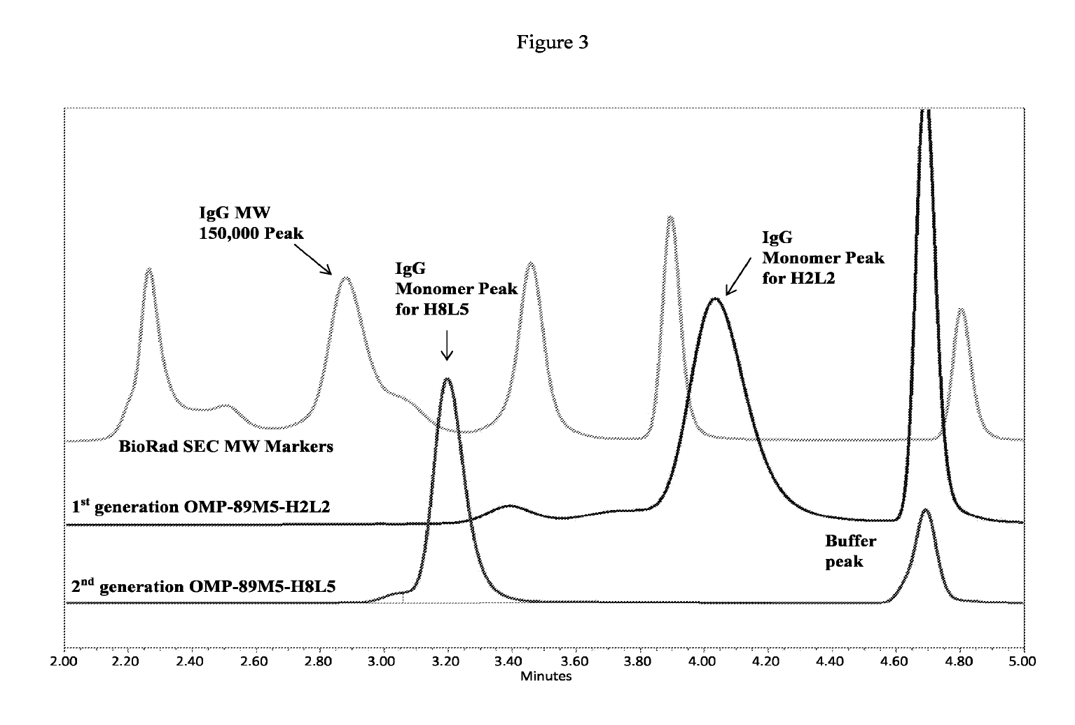

An overlay of the UV 215 nm wavelength traces for h89M5-H2L2 and h89M5-H8L5 are shown in FIG. 3. The figure also shows the UV trace for the MW standards, a mixture of proteins with MW ranging from 1,350 to 670,000 (BioRad Gel Filtration Standards, Cat. No. 151-1901). The IgG standard peak at approximately MW 150,000 is indicated. As mentioned in Example 1, first generation antibody h89R5-H2L2 was observed to have a major peak that eluted much later than the IgG standard peak, indicating a lower molecule weight than the expected 150,000. Second generation antibody h89R5-H8L5 was observed to have a major peak closer to the expected MW 150,000. Although the peak eluted later than the IgG standard peak, the h89M5-H8L5 peak elution time falls within a range observed for other antibodies, which can be explained, at least in part, by differences in each antibody's hydrodynamic radius. The elution profile for first generation antibody h89M5-H2L2 was significantly out of the normal range. It should be noted, that second generation antibody h89M5-H8L5 was characterized using other assays including mass spectrometry and non-reduced SDS-PAGE, and was shown to have a molecular weight of approximately 150,000. Importantly, second generation antibody h89M5-H8L5 was also shown to have increased solubility and could be formulated at higher concentrations for in vivo use.

### Example 4

### Binding Assay for Anti-RSPO1 Antibodies

High binding 96 well plates (Nalge Nunc International) were coated with 0.5 m/ml recombinant human RSPO1 (R&D Systems) in coating buffer (1× PBS, pH 7.4) overnight at 2-8° C. The plates were washed 3-6 times after each step with 300 μl/well of 1× PBS, 0.05% Tween-20, pH 7.4. After coating, the plates were incubated with 250 μl/well of blocking buffer (1× PBS, 0.1% gelatin, 0.1% TWEEN-20, pH 7.4) for 60 minutes ±10 minutes at room temperature (19-25° C.). Plates were washed and incubated with 100 μl/well of first generation humanized anti-RSPO1 antibody h89M5-H2L2 and second generation humanized antibody h89M5-H8L5 for 120 minutes ±10 minutes at room temperature. First generation humanized anti-RSPO1 antibody h89M5-H2L2 was use as the reference standard and the positive control. The antibodies were prepared at an initial concentration of 2.5 μg/ml and diluted 3-fold serially, ranging from 2.5 μg/ml to 0.014 ng/ml. Each sample was tested in duplicate and the experiment was run 3 times. To detect binding of the humanized anti-RSPO1 antibodies to RSPO1, goat F(ab)'2 anti-human IgG Fc-HRP conjugate (Jackson ImmunoResearch) was diluted in blocking buffer and added 100 μl/well for 60 minutes ±10 minutes at room temperature. The plate was developed with TMB substrate (Thermo Scientific Pierce) and the reaction stopped with 2M H₂SO₄. The binding of the antibodies to RSPO1 was measured by absorbance at 450 nm and 650 nm using an ELISA plate reader (Molecular Devices). The results were analyzed using a 4-parameter curve fit, and relative binding potency was determined by comparing EC₅₀ values of the reference to that of the samples.

The binding potency of second generation humanized anti-RSPO1 antibody h89M5-H8L5 was found to be better than (120% mean potency) the first generation antibody h89M5-H2L2.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

Following are the sequences disclosed in the application:

Human RSPO1 protein sequence with predicted signal
sequence underlined
(SEQ ID NO: 1)
MRLGLCVVALVLSWTHLTISSRGIKGKRQRRISAEGSQACAKGCELCSEV
NGCLKCSPKLFILLERNDIRQVGVCLPSCPPGYFDARNPDMNKCIKCKIE
HCEACFSHNFCTKCKEGLYLHKGRCYPACPEGSSAANGTMECSSPAQCEM
SEWSPWGPCSKKQQLCGFRRGSEERTRRVLHAPVGDHAACSDTKETRRCT
VRRVPCPEGQKRRKGGQGRRENANRNLARKESKEAGAGSRRRKGQQQQQQ
QGTVGPLTSAGPA
Human RSPO1 furin-like domain 1
(SEQ ID NO: 2)
AEGSQACAKGCELCSEVNGCLKCSPKLFILLERNDIRQVGVCLPSCPPGY
FD
Human RSPO1 furin-like domain 2
(SEQ ID NO: 3)
MNKCIKCKIEHCEACFSHNFCTKCKEGLYLHKGRCYPACPEGSSA
Human RSPO1 thrombospondin domain
(SEQ ID NO: 4)
QCEMSEWSPWGPCSKKQQLCGFRRGSEERTRRVLHAPVGDHAACSDTKET
RRCTVRRVPCP
Human RSPO1 amino acids 31-263
(SEQ ID NO: 5)
RISAEGSQACAKGCELCSEVNGCLKCSPKLFILLERNDIRQVGVCLPSCP
PGYFDARNPDMNKCIKCKIEHCEACFSHNFCTKCKEGLYLHKGRCYPACP
EGSSAANGTMECSSPAQCEMSEWSPWGPCSKKQQLCGFRRGSEERTRRVL
HAPVGDHAACSDTKETRRCTVRRVPCPEGQKRRKGGQGRRENANRNLARK
ESKEAGAGSRRRKGQQQQQQQGTVGPLTSAGPA
89M5 Heavy chain CDR1
(SEQ ID NO: 6)
TGYTMH
89M5 Heavy chain CDR2
(SEQ ID NO: 7)
GINPNNGGTTYNQNFKG
89M5 Heavy chain CDR3
(SEQ ID NO: 8)
KEFSDGYYFFAY
89M5 Light chain CDR1
(SEQ ID NO: 9)
KASQDVIFAVA
89M5 Light chain CDR2
(SEQ ID NO: 10)
WASTRHT
89M5 Light chain CDR3
(SEQ ID NO: 11)
QQHYSTPW
89M5 Heavy chain variable region
(SEQ ID NO: 12)
EVQLQQSGPELVKPGASVKISCKTSGYTFTGYTMHWVRQSHGKTLEWIGG
INPNNGGTTYNQNFKGKATLTVEKSSTTAYLELRSLTSEDSALYYCARKE
FSDGYYFFAYWGQGTLVTVSA
89M5 Light chain variable region
(SEQ ID NO: 13)
DIVMTQSHKFMSTSVGDRVNITCKASQDVIFAVAWYQQKPGQSPKLLIYW
ASTRHTGVPDRFTGSVSGTDYTLTISSVQAEDLALYYCQQHYSTPWTFGG
GTKLEIK
89M5 Heavy chain amino acid sequence with pre-
dicted signal sequence underlined
(SEQ ID NO: 14)
MGWSWIFLFLLSGTAGVLSEVQLQQSGPELVKPGASVKISCKTSGYTFTG
YTMHWVRQSHGKTLEWIGGINPNNGGTTYNQNFKGKATLTVEKSSTTAYL
ELRSLTSEDSALYYCARKEFSDGYYFFAYWGQGTLVTVSSAKTTPPSVYP
LAPGSAAQTNSMVTLGCLVKGYFPEPVTVTWNSGSLSSGVHTFPAVLQSD
LYTLSSSVTVPSSTWPSETVTCNVAHPASSTKVDKKIVPRDCGCKPCICT
VPEVSSVFIFPPKPKDVLTITLTPKVTCVVVDISKDDPEVQFSWFVDDVE
VHTAQTQPREEQFNSTFRSVSELPIMHQDWLNGKEFKCRVNSAAFPAPIE
KTISKTKGRPKAPQVYTIPPPKEQMAKDKVSLTCMITDFFPEDITVEWQW
NGQPAENYKNTQPIMDTDGSYFVYSKLNVQKSNWEAGNTFTCSVLHEGLH
NHHTEKSLSHSPGK
89M5 Heavy chain amino acid sequence without pre-
dicted signal sequence
(SEQ ID NO: 15)
EVQLQQSGPELVKPGASVKISCKTSGYTFTGYTMHWVRQSHGKTLEWIGG
INPNNGGTTYNQNFKGKATLTVEKSSTTAYLELRSLTSEDSALYYCARKE
FSDGYYFFAYWGQGTLVTVSSAKTTPPSVYPLAPGSAAQTNSMVTLGCLV
KGYFPEPVTVTWNSGSLSSGVHTFPAVLQSDLYTLSSSVTVPSSTWPSET
VTCNVAHPASSTKVDKKIVPRDCGCKPCICTVPEVSSVFIFPPKPKDVLT
ITLTPKVTCVVVDISKDDPEVQFSWFVDDVEVHTAQTQPREEQFNSTFRS
VSELPIMHQDWLNGKEFKCRVNSAAFPAPIEKTISKTKGRPKAPQVYTIP
PPKEQMAKDKVSLTCMITDFFPEDITVEWQWNGQPAENYKNTQPIMDTDG
SYFVYSKLNVQKSNWEAGNTFTCSVLHEGLHNHHTEKSLSHSPGK
89M5 Light chain amino acid sequence with pre-
dicted signal sequence underlined
(SEQ ID NO: 16)
MGFKMESQIQAFVFVFLWLSGVDGDIVMTQSHKFMSTSVGDRVNITCKAS
QDVIFAVAWYQQKPGQSPKLLIYWASTRHTGVPDRFTGSVSGTDYTLTIS
SVQAEDLALYYCQQHYSTPWTFGGGTKLEIKRADAAPTVSIFPPSSEQLT
SGGASVVCFLNNFYPKDINVKWKIDGSERQNGVLNSWTDQDSKDSTYSMS
STLTLTKDEYERHNSYTCEATHKTSTSPIVKSFNRNEC
89M5 Light chain amino acid sequence without pre-
dicted signal sequence
(SEQ ID NO: 17)
DIVMTQSHKFMSTSVGDRVNITCKASQDVIFAVAWYQQKPGQSPKLLIYW
ASTRHTGVPDRFTGSVSGTDYTLTISSVQAEDLALYYCQQHYSTPWTFGG
GTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC
89M5 Heavy chain variable region nucleotide
sequence
(SEQ ID NO: 18)
GAGGTCCAGCTGCAACAGTCTGGACCTGAGCTGGTGAAGCCTGGGGCTTC
AGTGAAGATATCCTGCAAGACTTCTGGATACACATTCACTGGATACACCA
TGCACTGGGTGAGGCAGAGCCATGGAAAGACCCTTGAGTGGATTGGAGGT
ATTAATCCTAACAATGGTGGTACTACTTACAACCAGAACTTCAAGGGCAA
GGCCACATTGACTGTAGAGAAGTCCTCCACCACAGCCTACTTGGAGCTCC
GCAGCCTGACATCTGAGGATTCTGCACTCTATTACTGTGCAAGAAAGGAG
TTCTCTGATGGTTACTACTTTTTTGCTTACTGGGGCCAAGGGACTCTGGT
CACTGTCTCTGCA
89M5 Light chain variable region nucleotide
sequence
(SEQ ID NO: 19)
GACATTGTGATGACCCAGTCTCACAAATTCATGTCCACATCAGTGGGAGA
CAGGGTCAACATCACCTGCAAGGCCAGTCAGGATGTGATTTTTGCTGTAG
CCTGGTATCAACAGAAACCAGGACAATCTCCTAAACTACTGATTTACTGG
GCATCCACCCGGCACACTGGAGTCCCTGATCGCTTCACAGGCAGTGTATC
TGGGACAGATTATACTCTCACCATCAGCAGTGTGCAGGCTGAAGACCTGG
CACTTTATTACTGTCAGCAACATTATAGCACTCCGTGGACGTTCGGTGGA
GGCACCAAGCTGGAAATCAAA
89M5 Heavy chain nucleotide sequence
(SEQ ID NO: 20)
ATGGGATGGAGCTGGATCTTTCTCTTTCTCCTGTCAGGAACTGCAGGTGT
CCTCTCTGAGGTCCAGCTGCAACAGTCTGGACCTGAGCTGGTGAAGCCTG
GGGCTTCAGTGAAGATATCCTGCAAGACTTCTGGATACACATTCACTGGA
TACACCATGCACTGGGTGAGGCAGAGCCATGGAAAGACCCTTGAGTGGAT
TGGAGGTATTAATCCTAACAATGGTGGTACTACTTACAACCAGAACTTCA
AGGGCAAGGCCACATTGACTGTAGAGAAGTCCTCCACCACAGCCTACTTG
GAGCTCCGCAGCCTGACATCTGAGGATTCTGCACTCTATTACTGTGCAAG
AAAGGAGTTCTCTGATGGTTACTACTTTTTTGCTTACTGGGGCCAAGGGA
CTCTGGTCACTGTCTCTTCAGCCAAAACGACACCCCCATCTGTCTATCCA
CTGGCCCCTGGATCTGCTGCCCAAACTAACTCCATGGTGACCCTGGGATG
CCTGGTCAAGGGCTATTTCCCTGAGCCAGTGACAGTGACCTGGAACTCTG
GATCCCTGTCCAGCGGTGTGCACACCTTCCCAGCTGTCCTGCAGTCTGAC
CTCTACACTCTGAGCAGCTCAGTGACTGTCCCCTCCAGCACCTGGCCCAG
CGAGACCGTCACCTGCAACGTTGCCCACCCGGCCAGCAGCACCAAGGTGG
ACAAGAAAATTGTGCCCAGGGATTGTGGTTGTAAGCCTTGCATATGTACA
GTCCCAGAAGTATCATCTGTCTTCATCTTCCCCCCAAAGCCCAAGGATGT
GCTCACCATTACTCTGACTCCTAAGGTCACGTGTGTTGTGGTAGACATCA
GCAAGGATGATCCCGAGGTCCAGTTCAGCTGGTTTGTAGATGATGTGGAG
GTGCACACAGCTCAGACGCAACCCCGGGAGGAGCAGTTCAACAGCACTTT
CCGCTCAGTCAGTGAACTTCCCATCATGCACCAGGACTGGCTCAATGGCA
AGGAGTTCAAATGCAGGGTCAACAGTGCAGCTTTCCCTGCCCCCATCGAG
AAAACCATCTCCAAAACCAAAGGCAGACCGAAGGCTCCACAGGTGTACAC
CATTCCACCTCCCAAGGAGCAGATGGCCAAGGATAAAGTCAGTCTGACCT
GCATGATAACAGACTTCTTCCCTGAAGACATTACTGTGGAGTGGCAGTGG
AATGGGCAGCCAGCGGAGAACTACAAGAACACTCAGCCCATCATGGACAC
AGATGGCTCTTACTTCGTCTACAGCAAGCTCAATGTGCAGAAGAGCAACT
GGGAGGCAGGAAATACTTTCACCTGCTCTGTGTTACATGAGGGCCTGCAC
AACCACCATACTGAGAAGAGCCTCTCCCACTCTCCTGGTAAATGATAA
89M5 Light chain nucleotide sequence
(SEQ ID NO: 21)
ATGGGCTTCAAGATGGAGTCACAGATTCAGGCATTTGTATTCGTGTTTCT
CTGGTTGTCTGGTGTTGACGGAGACATTGTGATGACCCAGTCTCACAAAT
TCATGTCCACATCAGTGGGAGACAGGGTCAACATCACCTGCAAGGCCAGT
CAGGATGTGATTTTTGCTGTAGCCTGGTATCAACAGAAACCAGGACAATC
TCCTAAACTACTGATTTACTGGGCATCCACCCGGCACACTGGAGTCCCTG
ATCGCTTCACAGGCAGTGTATCTGGGACAGATTATACTCTCACCATCAGC
AGTGTGCAGGCTGAAGACCTGGCACTTTATTACTGTCAGCAACATTATAG
CACTCCGTGGACGTTCGGTGGAGGCACCAAGCTGGAAATCAAACGGGCTG
ATGCTGCACCAACTGTATCCATCTTCCCACCATCCAGTGAGCAGTTAACA
TCTGGAGGTGCCTCAGTCGTGTGCTTCTTGAACAACTTCTACCCCAAAGA
CATCAATGTCAAGTGGAAGATTGATGGCAGTGAACGACAAAATGGCGTCC
TGAACAGTTGGACTGATCAGGACAGCAAAGACAGCACCTACAGCATGAGC
AGCACCCTCACGTTGACCAAGGACGAGTATGAACGACATAACAGCTATAC
CTGTGAGGCCACTCACAAGACATCAACTTCACCCATTGTCAAGAGCTTCA
ACAGGAATGAGTGTTAGTGA
Humanized
h89M5-H2L2 Heavy chain variable region amino acid
sequence
(SEQ ID NO: 22)
QVQLVQSGAEVKKPGASVKVSCKTSGYTFTGYTMHWVRQAPGQRLEWMGG
INPNNGGTTYNQNFKGRVTITRDTSASTAYMELSSLRSEDTAVYYCARKE
FSDGYYFFAYWGQGTLVTVSS
h89M5-H2L2 Light chain variable region amino acid
sequence
(SEQ ID NO: 23)
DIQMTQSPSSLSASVGDRVTITCKASQDVIFAVAWYQQKPGKAPKLLIYW
ASTRHTGVPSRFSGSGSGTDYTLTISSLQPEDFATYYCQQHYSTPWTFGG
GTKVEIK
h89M5-H2L2 Heavy chain amino acid sequence with
predicted signal sequence underlined
(SEQ ID NO: 24)
MDWTWRILFLVAAATGAHSQVQLVQSGAEVKKPGASVKVSCKTSGYTFTG
YTMHWVRQAPGQRLEWMGGINPNNGGTTYNQNFKGRVTITRDTSASTAYM
ELSSLRSEDTAVYYCARKEFSDGYYFFAYWGQGTLVTVSSASTKGPSVFP
LAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDG
VEVHNAKTKPREEQFNSTFRVVSVLTVVHQDWLNGKEYKCKVSNKGLPAP
IEKTISKTKGQPREPQVYTLPPSREEMTKNQVSLTCLVKGFYPSDIAVEW
ESNGQPENNYKTTPPMLDSDGSFFLYSKLTVDKSRWQQGNVFSCSVMHEA
LHNHYTQKSLSLSPGK
h89M5-H2L2 Heavy chain amino acid sequence without
predicted signal sequence
(SEQ ID NO: 25)
QVQLVQSGAEVKKPGASVKVSCKTSGYTFTGYTMHWVRQAPGQRLEWMGG
INPNNGGTTYNQNFKGRVTITRDTSASTAYMELSSLRSEDTAVYYCARKE
FSDGYYFFAYWGQGTLVTVSSASTKGPSVFPLAPCSRSTSESTAALGCLV
KDYFPEPVTVSWNSGALTSGVHTFPAVLQSSGLYSLSSVVTVPSSNFGTQ
TYTCNVDHKPSNTKVDKTVERKCCVECPPCPAPPVAGPSVFLFPPKPKDT
LMISRTPEVTCVVVDVSHEDPEVQFNWYVDGVEVHNAKTKPREEQFNSTF
RVVSVLTVVHQDWLNGKEYKCKVSNKGLPAPIEKTISKTKGQPREPQVYT
LPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK
h89M5-H2L2 Light chain amino acid sequence with
predicted signal sequence underlined
(SEQ ID NO: 26)
MDMRVPAQLLGLLLLWLRGARCDIQMTQSPSSLSASVGDRVTITCKASQD
VIFAVAWYQQKPGKAPKLLIYWASTRHTGVPSRFSGSGSGTDYTLTISSL
QPEDFATYYCQQHYSTPWTFGGGTKVEIKRTVAAPSVFIFPPSDEQLKSG
TASVVCLLNNFYPREAKVQWKVDNALQSGNSQESVTEQDSKDSTYSLSNT
LTLSKADYEKHKVYACEVTHQGLSSPVTKSFNRGEC
h89M5-H2L2 Light chain amino acid sequence without
predicted signal sequence underlined
(SEQ ID NO: 27)
DIQMTQSPSSLSASVGDRVTITCKASQDVIFAVAWYQQKPGKAPKLLIYW
ASTRHTGVPSRFSGSGSGTDYTLTISSLQPEDFATYYCQQHYSTPWTFGG
GTKVEIKRTVAAPSVFIFPPSDEQLKSGTASVVCLLNNFYPREAKVQWKV
DNALQSGNSQESVTEQDSKDSTYSLSNTLTLSKADYEKHKVYACEVTHQG
LSSPVTKSFNRGEC
h89M5-H2L2 Heavy chain variable region nucleotide
sequence
(SEQ ID NO: 28)
CAGGTCCAGCTCGTGCAGTCTGGGGCTGAGGTGAAGAAGCCTGGGGCCTC
TGTGAAGGTTTCCTGCAAGACTTCTGGATACACCTTCACTGGATACACCA
TGCACTGGGTTAGACAGGCCCCCGGACAAAGGCTGGAGTGGATGGGAGGT
ATTAATCCTAACAATGGTGGTACTACTTACAACCAGAACTTCAAGGGCAG
AGTCACCATTACCAGGGACACATCCGCAAGCACAGCCTACATGGAGCTGT
CCAGCCTGAGATCTGAAGACACAGCTGTGTATTACTGTGCAAGAAAGGAG
TTCTCTGATGGATACTACTTTTTTGCTTACTGGGGCCAAGGGACCCTGGT
CACCGTCAGCTCA
h89M5-H2L2 Light chain variable region nucleotide
sequence
(SEQ ID NO: 29)
GACATCCAGATGACCCAGTCTCCATCCTCCCTGTCTGCATCTGTCGGAGA
CAGAGTCACCATCACTTGCAAGGCCTCCCAGGATGTGATTTTTGCTGTTG
CCTGGTATCAGCAGAAACCAGGGAAAGCCCCTAAGCTCCTGATCTATTGG
GCATCCACCCGGCACACTGGGGTCCCATCAAGGTTCAGTGGCAGTGGATC
TGGGACAGATTACACTCTCACCATCAGCAGTCTGCAACCTGAAGATTTTG
CAACTTACTACTGTCAGCAACATTATAGCACTCCTTGGACTTTCGGCGGA
GGGACCAAGGTGGAGATCAAA
h89M5-H2L2 Heavy chain nucleotide sequence
(SEQ ID NO: 30)
ATGGACTGGACCTGGAGGATACTCTTTCTCGTGGCAGCAGCCACAGGAGC
CCACTCCCAGGTCCAGCTCGTGCAGTCTGGGGCTGAGGTGAAGAAGCCTG
GGGCCTCTGTGAAGGTTTCCTGCAAGACTTCTGGATACACCTTCACTGGA
TACACCATGCACTGGGTTAGACAGGCCCCCGGACAAAGGCTGGAGTGGAT
GGGAGGTATTAATCCTAACAATGGTGGTACTACTTACAACCAGAACTTCA
AGGGCAGAGTCACCATTACCAGGGACACATCCGCAAGCACAGCCTACATG
GAGCTGTCCAGCCTGAGATCTGAAGACACAGCTGTGTATTACTGTGCAAG
AAAGGAGTTCTCTGATGGATACTACTTTTTTGCTTACTGGGGCCAAGGGA
CCCTGGTCACCGTCAGCTCAGCCAGCACAAAGGGCCCTAGCGTCTTCCCT
CTGGCTCCCTGCAGCAGGAGCACCAGCGAGAGCACAGCCGCCCTGGGCTG
CCTGGTCAAGGACTACTTCCCCGAACCGGTGACGGTGTCGTGGAACTCAG
GCGCTCTGACCAGCGGCGTGCACACCTTCCCAGCTGTCCTACAGTCCTCA
GGACTCTACTCCCTCAGCAGCGTGGTGACCGTGCCCTCCAGCAACTTCGG
CACCCAGACCTACACCTGCAACGTAGATCACAAGCCCAGCAACACCAAGG
TGGACAAGACAGTTGAGCGCAAATGTTGTGTCGAGTGCCCACCGTGCCCA
GCACCACCTGTGGCAGGACCGTCAGTCTTCCTCTTCCCCCCAAAACCCAA
GGACACCCTCATGATCTCCCGGACCCCTGAGGTCACGTGCGTGGTGGTGG
ACGTGAGCCACGAAGACCCCGAGGTCCAGTTCAACTGGTACGTGGACGGC
GTGGAGGTGCATAATGCCAAGACAAAGCCACGGGAGGAGCAGTTCAACAG
CACGTTCCGTGTGGTCAGCGTCCTCACCGTTGTGCACCAGGACTGGCTGA
ACGGCAAGGAGTACAAGTGCAAGGTCTCCAACAAAGGCCTCCCAGCCCCC
ATCGAGAAAACCATCTCCAAAACCAAAGGGCAGCCCCGAGAACCACAGGT
GTACACCCTGCCCCCATCCCGGGAGGAGATGACCAAGAACCAGGTCAGCC
TGACCTGCCTGGTCAAAGGCTTCTACCCCAGCGACATCGCCGTGGAGTGG
GAGAGCAATGGGCAGCCGGAGAACAACTACAAGACCACACCTCCCATGCT
GGACTCCGACGGCTCCTTCTTCCTCTACAGCAAGCTCACCGTGGACAAGA
GCAGGTGGCAGCAGGGGAACGTCTTCTCATGCTCCGTGATGCATGAGGCT
CTGCACAACCACTACACGCAGAAGAGCCTCTCCCTGTCTCCGGGTAAATG
A
h89M5-H2L2 Light chain nucleotide sequence
(SEQ ID NO: 31)
ATGGACATGAGGGTCCCCGCACAGCTCCTGGGGCTCCTGCTCCTCTGGCT
CCGGGGTGCCAGATGTGACATCCAGATGACCCAGTCTCCATCCTCCCTGT
CTGCATCTGTCGGAGACAGAGTCACCATCACTTGCAAGGCCTCCCAGGAT
GTGATTTTTGCTGTTGCCTGGTATCAGCAGAAACCAGGGAAAGCCCCTAA
GCTCCTGATCTATTGGGCATCCACCCGGCACACTGGGGTCCCATCAAGGT
TCAGTGGCAGTGGATCTGGGACAGATTACACTCTCACCATCAGCAGTCTG
CAACCTGAAGATTTTGCAACTTACTACTGTCAGCAACATTATAGCACTCC
TTGGACTTTCGGCGGAGGGACCAAGGTGGAGATCAAACGGACTGTGGCTG
CACCATCTGTCTTCATCTTCCCTCCATCTGATGAGCAGTTGAAATCTGGA
ACTGCCTCTGTTGTGTGCCTGCTGAATAACTTCTATCCCAGAGAGGCCAA
AGTCCAGTGGAAGGTGGATAACGCCCTCCAATCCGGTAACTCCCAGGAGA
GTGTCACAGAGCAGGACAGCAAGGACAGCACCTACAGCCTCAGCAACACC
CTGACACTGAGCAAAGCAGACTACGAGAAACACAAAGTCTACGCCTGCGA
AGTCACCCATCAGGGCCTGAGCTCCCCCGTCACAAAGAGCTTCAACAGGG
GAGAGTGCTAA
h89M5-H8L5 Heavy chain variable region amino acid
sequence
(SEQ ID NO: 32)
EVQLVQSGAEVKKPGESLRISCKGSGYSFTGYTMHWVRQMPGKGLEWMGG
INPNNGGTTYNQNFKGHVTISADKSISTAYLQWSSLKASDTAMYYCARKE
FSDGYYFFAYWGQGTLVTVSS
h89M5-H8L5 Light chain variable region amino acid
sequence
(SEQ ID NO: 33)
DIVMTQSPDSLAVSLGERATINCKASQDVIFAVAWYQQKPGQPPKLLIYW
ASTRHTGVPDRFSGSGSGTDFTLTISSLQAEDVAVYYCQQHYSTPWTFGG
GTKVEIK
h89M5-H8L5 Heavy chain amino acid sequence with
predicted signal sequence underlined
(SEQ ID NO: 34)
MDWTWRILFLVAAATGAHSEVQLVQSGAEVKKPGESLRISCKGSGYSFTG
YTMHWVRQMPGKGLEWMGGINPNNGGTTYNQNFKGHVTISADKSISTAYL
QWSSLKASDTAMYYCARKEFSDGYYFFAYWGQGTLVTVSSASTKGPSVFP
LAPSSKSTSGGTAALGCLVKDYFPEPVTVSWNSGALTSGVHTFPAVLQSS
GLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKA
LPAPIEKTISKAKGQPREPQVYTLPPSREEMTKNQVSLTCLVKGFYPSDI
AVEWESNGQPENNYKTTPPVLDSDGSFFLYSKLTVDKSRWQQGNVFSCSV
MHEALHNHYTQKSLSLSPGK
h89M5-H8L5 Heavy chain amino acid sequence without
predicted signal sequence
(SEQ ID NO: 35)
EVQLVQSGAEVKKPGESLRISCKGSGYSFTGYTMHWVRQMPGKGLEWMGG
INPNNGGTTYNQNFKGHVTISADKSISTAYLQWSSLKASDTAMYYCARKE
FSDGYYFFAYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
h89M5-H8L5 Light chain amino acid sequence with
predicted signal sequence underlined
(SEQ ID NO: 36)
MVLQTQVFISLLLWISGAYGDIVMTQSPDSLAVSLGERATINCKASQDVI
FAVAWYQQKPGQPPKLLIYWASTRHTGVPDRFSGSGSGTDFTLTISSLQA
EDVAVYYCQQHYSTPWTFGGGTKVEIKRTVAAPSVFIFPPSDEQLKSGTA
SVVCLLNNFYPREAKVQWKVDNALQSGNSQESVTEQDSKDSTYSLSNTLT
LSKADYEKHKVYACEVTHQGLSSPVTKSFNRGEC
h89M5-H8L5 Light chain amino acid sequence without
predicted signal sequence
(SEQ ID NO: 37)
DIVMTQSPDSLAVSLGERATINCKASQDVIFAVAWYQQKPGQPPKLLIYW
ASTRHTGVPDRFSGSGSGTDFTLTISSLQAEDVAVYYCQQHYSTPWTFGG
GTKVEIKRTVAAPSVFIFPPSDEQLKSGTASVVCLLNNFYPREAKVQWKV
DNALQSGNSQESVTEQDSKDSTYSLSNTLTLSKADYEKHKVYACEVTHQG
LSSPVTKSFNRGEC
h89M5-H8L5 Heavy chain variable region nucleotide
sequence
(SEQ ID NO: 38)
GAAGTGCAGCTGGTGCAGTCTGGAGCAGAGGTCAAAAAGCCCGGGGAGTC
TCTGAGGATCTCCTGCAAGGGTTCTGGATACAGCTTTACTGGATACACCA
TGCACTGGGTGCGCCAGATGCCCGGGAAAGGACTGGAGTGGATGGGGGGT
ATTAATCCTAACAATGGTGGTACTACTTACAACCAGAACTTCAAGGGCCA
CGTCACCATCTCAGCTGACAAGTCCATCAGCACTGCCTACCTGCAATGGA
GCAGCCTGAAGGCTTCTGACACCGCCATGTATTACTGTGCAAGAAAGGAG
TTCTCTGATGGATACTACTTTTTTGCTTACTGGGGCCAAGGGACCCTGGT
GACCGTCAGCTCA
h89M5-H8L5 Light chain variable region nucleotide
sequence
(SEQ ID NO: 39)
GACATCGTGATGACCCAGTCTCCAGACTCCCTGGCTGTGTCTCTGGGCGA
GAGGGCCACCATCAACTGCAAGGCTTCCCAGGACGTGATTTTTGCTGTTG
CCTGGTATCAGCAGAAACCAGGACAGCCTCCTAAGCTGCTCATTTACTGG
GCATCTACCCGCCACACTGGGGTCCCTGACCGCTTCAGTGGCAGCGGGTC
TGGGACAGATTTCACTCTCACCATCAGCAGCCTCCAGGCTGAAGATGTGG
CAGTTTATTACTGTCAGCAACATTATAGCACTCCTTGGACTTTCGGCGGA
GGGACCAAGGTGGAGATCAAA
h89M5-H8L5 Heavy chain nucleotide sequence
(SEQ ID NO: 40)
ATGGACTGGACCTGGAGGATACTCTTTCTCGTGGCGGCAGCCACAGGAGC
CCACTCCGAAGTGCAGCTGGTGCAGTCTGGAGCAGAGGTCAAAAAGCCCG
GGGAGTCTCTGAGGATCTCCTGCAAGGGTTCTGGATACAGCTTTACTGGA
TACACCATGCACTGGGTGCGCCAGATGCCCGGGAAAGGACTGGAGTGGAT
GGGGGGTATTAATCCTAACAATGGTGGTACTACTTACAACCAGAACTTCA
AGGGCCACGTCACCATCTCAGCTGACAAGTCCATCAGCACTGCCTACCTG
CAATGGAGCAGCCTGAAGGCTTCTGACACCGCCATGTATTACTGTGCAAG
AAAGGAGTTCTCTGATGGATACTACTTTTTTGCTTACTGGGGCCAAGGGA
CCCTGGTGACCGTCAGCTCAGCCAGCACAAAGGGCCCCTCCGTGTTCCCT
CTGGCCCCTTCCTCCAAGTCCACCTCCGGCGGCACCGCCGCTCTGGGCTG
CCTGGTGAAGGACTACTTCCCTGAGCCTGTGACCGTGTCCTGGAACTCTG
GCGCACTGACCTCTGGCGTGCACACCTTCCCAGCCGTGCTCCAGTCCTCC
GGCCTGTACTCCCTGTCCTCCGTCGTCACCGTGCCTTCCTCCTCCCTGGG
CACCCAGACCTACATCTGCAACGTGAACCACAAGCCTTCCAACACAAAGG
TGGACAAGCGGGTGGAGCCTAAGTCCTGCGACAAGACCCACACCTGCCCT
CCCTGCCCTGCCCCTGAGCTGCTGGGCGGACCTTCCGTGTTCCTGTTCCC
TCCTAAGCCTAAGGACACCCTGATGATCTCCCGGACCCCTGAAGTGACAT
GCGTGGTGGTGGACGTGTCCCACGAGGACCCTGAGGTGAAGTTCAACTGG
TATGTGGACGGCGTGGAGGTGCACAACGCTAAGACCAAGCCGAGGGAGGA
GCAGTACAACTCCACCTACCGGGTGGTGTCTGTGCTGACCGTGCTGCACC
AGGACTGGCTGAACGGCAAAGAATACAAGTGCAAGGTCTCCAACAAGGCC
CTGCCCGCTCCCATCGAGAAAACCATCAGCAAGGCAAAGGGCCAGCCTCG
CGAGCCTCAGGTGTACACCCTGCCACCCAGCCGGGAGGAGATGACCAAGA
ACCAGGTGTCCCTGACCTGTCTGGTGAAGGGCTTTTACCCTTCCGATATT
GCCGTGGAGTGGGAGTCTAACGGCCAGCCCGAGAACAACTACAAGACCAC
CCCTCCTGTGCTGGACTCCGACGGCTCCTTCTTCCTGTACTCCAAGCTGA
CCGTGGACAAGTCCCGGTGGCAGCAGGGCAACGTGTTCTCCTGCTCCGTG
ATGCACGAGGCCCTGCACAACCACTACACCCAGAAGAGCCTGTCTCTGTC
TCCTGGCAAGTGA
h89M5-H8L5 Light chain nucleotide sequence
(SEQ ID NO: 41)
ATGGTGCTCCAGACCCAGGTCTTCATTTCTCTGCTCCTCTGGATCTCTGG
TGCCTACGGGGACATCGTGATGACCCAGTCTCCAGACTCCCTGGCTGTGT
CTCTGGGCGAGAGGGCCACCATCAACTGCAAGGCTTCCCAGGACGTGATT
TTTGCTGTTGCCTGGTATCAGCAGAAACCAGGACAGCCTCCTAAGCTGCT
CATTTACTGGGCATCTACCCGCCACACTGGGGTCCCTGACCGCTTCAGTG
GCAGCGGGTCTGGGACAGATTTCACTCTCACCATCAGCAGCCTCCAGGCT
GAAGATGTGGCAGTTTATTACTGTCAGCAACATTATAGCACTCCTTGGAC
TTTCGGCGGAGGGACCAAGGTGGAGATCAAACGGACTGTGGCTGCACCAT
CTGTCTTCATCTTCCCTCCATCTGATGAGCAGTTGAAATCTGGAACTGCC
TCTGTTGTGTGCCTGCTGAATAACTTCTATCCCAGAGAGGCCAAAGTCCA
GTGGAAGGTGGATAACGCCCTCCAATCCGGTAACTCCCAGGAGAGTGTCA
CAGAGCAGGACAGCAAGGACAGCACCTACAGCCTCAGCAACACCCTGACA
CTGAGCAAAGCAGACTACGAGAAACACAAAGTCTATGCCTGCGAAGTCAC
CCATCAGGGCCTGTCTTCCCCCGTCACAAAGAGCTTCAACAGGGGAGAGT
GCTAA

What is claimed is:

1. An isolated antibody that specifically binds human R-spondin 1 (RSPO1), which comprises a heavy chain variable region comprising SEQ ID NO:32 and a light chain variable region comprising SEQ ID NO:33.

2. The antibody of claim 1, wherein the heavy chain variable region consists of SEQ ID NO:32 and the light chain variable region consists SEQ ID NO:33.

3. An isolated antibody that specifically binds human R-spondin 1 (RSPO1), which comprises a heavy chain variable region having at least 90% sequence identity to SEQ ID NO:32 and a light chain variable region having at least 90% sequence identity to SEQ ID NO:33, wherein the antibody comprises a heavy chain CDR1 comprising TGYTMH (SEQ ID NO:6), a heavy chain CDR2 comprising GINPNNGGTTYNQNFKG (SEQ ID NO:7), and a heavy chain CDR3 comprising KEFSDGYYFFAY (SEQ ID NO:8), a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11).

4. The antibody of any one of claims 1-3, which is an IgG1 antibody or an IgG2 antibody.

5. The antibody of any one of claims 1-3, which is an antibody fragment comprising an antigen binding site.

6. An isolated antibody that specifically binds human RSPO1, which comprises a heavy chain comprising SEQ ID NO:35 and a light chain comprising SEQ ID NO:37.

7. An isolated antibody that specifically binds human R-spondin 1 (RSPO1), which comprises a heavy chain having at least 90% sequence identity to SEQ ID NO:35 and a light chain having at least 90% sequence identity to SEQ ID NO:37, wherein the antibody comprises a heavy chain CDR1 comprising TGYTMH (SEQ ID NO:6), a heavy chain CDR2 comprising GINPNNGGTTYNQNFKG (SEQ ID NO:7), and a heavy chain CDR3 comprising KEFSDGYYFFAY (SEQ ID NO:8), a light chain CDR1 comprising KASQDVIFAVA (SEQ ID NO:9), a light chain CDR2 comprising WASTRHT (SEQ ID NO:10), and a light chain CDR3 comprising QQHYSTPW (SEQ ID NO:11).

8. The antibody of any one of claims 1-7, which is a recombinant antibody, a monoclonal antibody, a chimeric antibody, or a bispecific antibody.

9. The antibody of any one of claim 1-7, which is antibody h89M5-H8L5.

10. An antibody comprising the heavy chain variable region encoded by the plasmid deposited with ATCC as PTA-121494.

11. An antibody comprising the heavy chain encoded by the plasmid deposited with ATCC as PTA-121494.

12. An antibody comprising the light chain variable region encoded by the plasmid deposited with ATCC as PTA-121495.

13. An antibody comprising the light chain encoded by the plasmid deposited with ATCC as PTA-121495.

14. An antibody comprising the heavy chain variable region encoded by the plasmid deposited with ATCC as PTA-121494 and the light chain variable region encoded by the plasmid deposited with ATCC as PTA-121495.

15. An antibody comprising the heavy chain encoded by the plasmid deposited with ATCC as PTA-121494 and the light chain encoded by the plasmid deposited with ATCC as PTA-121495.

16. The antibody of any one of claims 1-15, which inhibits binding of RSPO1 to at least one leucine-rich repeat containing G protein coupled receptor (LGR).

17. The antibody of claim 16, wherein the LGR is selected from the group consisting of LGR4, LGRS, and LGR6.

18. The antibody of claim 17, wherein the LGR is LGRS.

19. The antibody of any one of claims 1-18, which:
(a) inhibits RSPO1 signaling;
(b) inhibits activation of β-catenin; and/or
(c) inhibits β-catenin signaling.

20. The antibody of any one of claims 1-19, which inhibits tumor growth.

21. A polypeptide comprising a sequence selected from the group consisting of: SEQ ID NO:32, SEQ ID NO:33, SEQ ID NO:34, SEQ ID NO:35, SEQ ID NO:36, and SEQ ID NO:37.

22. The polypeptide of claim 21, which is an antibody.

23. A pharmaceutical composition comprising the antibody or polypeptide of any one of claims 1-22 and a pharmaceutically acceptable carrier.

24. A cell comprising or producing the antibody or polypeptide of any one of claims 1-22.

25. An isolated polynucleotide molecule comprising a nucleotide sequence that encodes an antibody or polypeptide of any one of claims 1-22.

26. An isolated polynucleotide molecule comprising a nucleotide sequence selected from the group consisting of: SEQ ID NO:38, SEQ ID NO:39, SEQ ID NO:40, and SEQ ID NO:41.

27. A vector comprising the polynucleotide of claim 25 or claim 26.

28. A cell comprising the polynucleotide of claim 25 or claim 26 or the vector of claim 27.

29. A method of inhibiting growth of a tumor, wherein the method comprises contacting the tumor with an effective amount of an antibody of any one of claims 1-15.

30. A method of inhibiting growth of a tumor in a subject, wherein the method comprises administering to the subject a therapeutically effective amount of an antibody of any one of claims 1-15.

31. A method of inhibiting β-catenin signaling in a cell, comprising contacting the cell with an effective amount of an antibody of any one of claims 1-15.

32. The method of claim 31, wherein the cell is a tumor cell.

33. The method of any one of claim 29, 30, or 32, wherein the tumor is selected from the group consisting of colorectal tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

34. A method of treating cancer in a subject, wherein the method comprises administering to the subject a therapeutically effective amount of an antibody of any one of claims 1-15.

35. The method of claim 34, wherein the cancer is selected from the group consisting of colorectal cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer.

36. The method of any one of claim 29, 30, or 32-35, wherein the tumor or the cancer expresses elevated levels of RSPO1 as compared to a predetermined level of RSPO1.

37. The method of any one of claim 29, 30, or 32-36, wherein the subject has had a tumor or a cancer removed.

38. The method of any one of claim 29, 30, or 32-37, further comprising a step of determining the level of RSPO1 expression in the tumor or cancer.

39. The method of claim 38, wherein determining the level of RSPO1 expression is done prior to treatment or contact with the antibody.

40. A method of treating a disease in a subject wherein the disease is associated with activation of β-catenin, comprising administering a therapeutically effective amount of an antibody of any one of claims 1-15.

41. The method according to any one of claims 29-40, which further comprises administering at least one additional therapeutic agent.

42. The method of claim 41, wherein the additional therapeutic agent is a chemotherapeutic agent.

43. The method of claim 41, wherein the additional therapeutic agent is an angiogenesis inhibitor.

44. The method of claim 41, wherein the additional therapeutic agent is a second anti-RSPO antibody.

45. The method of any one of claim 29, 30, or 32-44, wherein the subject is human.

46. A method of selecting a human subject for treatment with an antibody that binds RSPO1, comprising: determining if the subject has a tumor that has an elevated expression level of RSPO1, wherein if the tumor has an elevated expression level of RSPO1 the subject is selected for treatment with an antibody of any one of claims 1-15.

47. The method of claim 46, wherein the expression level of RSPO1 is determined in a sample by a PCR-based assay, microarray analysis, or nucleotide sequencing.

48. The method of claim 47, wherein the sample is a fresh tumor sample, a frozen tumor sample, or a formalin-fixed paraffin-embedded sample.

49. The use of the antibody of any one of claims 1-15 for inhibiting growth of a tumor.

50. The use of the antibody of any one of claims 1-15 for inhibiting β-catenin signaling in a cell.

51. The use of the antibody of claim 50, wherein the cell is a tumor cell.

52. The use of the antibody of claim 49 or claim 51, wherein the tumor is selected from the group consisting of colorectal tumor, ovarian tumor, pancreatic tumor, lung tumor, liver tumor, breast tumor, kidney tumor, prostate tumor, gastrointestinal tumor, melanoma, cervical tumor, bladder tumor, glioblastoma, and head and neck tumor.

53. The use of the antibody of any one of claims 1-15 for treating cancer.

54. The use of the antibody of claim 53, wherein the cancer is selected from the group consisting of colorectal cancer, ovarian cancer, pancreatic cancer, lung cancer, liver cancer, breast cancer, kidney cancer, prostate cancer, gastrointestinal cancer, melanoma, cervical cancer, bladder cancer, glioblastoma, and head and neck cancer.

55. The use of the antibody of any one of claim 49 or 51-54, wherein the tumor or the cancer expresses elevated levels of RSPO1 as compared to a predetermined level of RSPO1.

56. The use of the antibody of any one of claim 49 or 51-54, wherein the expression level of RSPO1 in the tumor or cancer is determined.

57. The use of the antibody of any one of claims 1-15 for treating a disease associated with activation of β-catenin.

58. The use of the antibody of any one of claims 49-57, which comprises use of the antibody in combination with at least one additional therapeutic agent.

59. The use of the antibody of claim 58, wherein the additional therapeutic agent is a chemotherapeutic agent.

60. The use of the antibody of claim 58, wherein the additional therapeutic agent is an angiogenesis inhibitor.

61. The use of the antibody of claim 58, wherein the additional therapeutic agent is a second anti-RSPO antibody.

62. The use of the antibody of claim 56, wherein the expression level of RSPO1 is determined in a sample by a PCR-based assay, microarray analysis, or nucleotide sequencing.

63. The use of the antibody of claim 62, wherein the sample is a fresh tumor sample, a frozen tumor sample, or a formalin-fixed paraffin-embedded sample.

64. A plasmid deposited with ATCC and assigned designation number PTA-121494.

65. A plasmid deposited with ATCC and assigned designation number PTA-121495.


SEQUENCE LISTING (41 sequences)

SEQ ID NO: 1 - protein, 263 aa, Artificial Sequence
  Human RSPO1 protein sequence
  SIGNAL (1)..(20): predicted signal sequence
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 2 - protein, 52 aa, Artificial Sequence
  Human RSPO1 furin-like domain 1
AEGSQACAKGCELCSEVNGCLKCSPKLFILLERNDIRQVGVCLPSCPPGYFD

SEQ ID NO: 3 - protein, 45 aa, Artificial Sequence
  Human RSPO1 furin-like domain 2
MNKCIKCKIEHCEACFSHNFCTKCKEGLYLHKGRCYPACPEGSSA

SEQ ID NO: 4 - protein, 61 aa, Artificial Sequence
  Human RSPO1 thrombospondin domain
QCEMSEWSPWGPCSKKQQLCGFRRGSEERTRRVLHAPVGDHAACSDTKETRRCTVRRVPCP

SEQ ID NO: 5 - protein, 233 aa, Artificial Sequence
  Human RSPO1 amino acids 31-263
RISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 6 - protein, 6 aa, Artificial Sequence
  89M5 Heavy chain CDR1
TGYTMH

SEQ ID NO: 7 - protein, 17 aa, Artificial Sequence
  89M5 Heavy chain CDR2
GINPNNGGTTYNQNFKG

SEQ ID NO: 8 - protein, 12 aa, Artificial Sequence
  89M5 Heavy chain CDR3
KEFSDGYYFFAY

SEQ ID NO: 9 - protein, 11 aa, Artificial Sequence
  89M5 Light chain CDR1
KASQDVIFAVA

SEQ ID NO: 10 - protein, 7 aa, Artificial Sequence
  89M5 Light chain CDR2
WASTRHT

SEQ ID NO: 11 - protein, 8 aa, Artificial Sequence
  89M5 Light chain CDR3
QQHYSTPW

SEQ ID NO: 12 - protein, 121 aa, Artificial Sequence
  89M5 Heavy chain variable region
EVQLQQSGPELVKPGASVKISCKTSGYTFTGYTMHWVRQSHGKTLEWIGGINPNNGGTTYNQNFKGKATLTVEKSSTTAYLELRSLTSEDSALYYCARKEFSDGYYFFAYWGQGTLVTVSA

SEQ ID NO: 13 - protein, 107 aa, Artificial Sequence
  89M5 Light chain variable region
DIVMTQSHKFMSTSVGDRVNITCKASQDVIFAVAWYQQKPGQSPKLLIYWASTRHTGVPDRFTGSVSGTDYTLTISSVQAEDLALYYCQQHYSTPWTFGGGTKLEIK

SEQ ID NO: 14 - protein, 464 aa, Artificial Sequence
  89M5 Heavy chain amino acid sequence
  SIGNAL (1)..(19): predicted signal sequence
MGWSWIFLFLLSGTAGVLSEVQLQQSGPELVKPGASVKISCKTSGYTFTGYTMHWVRQSHGKTLEWIGGINPNNGGTTYNQNFKGKATLTVEKSSTTAYLELRSLTSEDSALYYCARKEFSDGYYFFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 15 - protein, 445 aa, Artificial Sequence
  89M5 Heavy chain amino acid sequence without predicted signal sequence
EVQLQQSGPELVKPGASVKISCKTSGYTFTGYTMHWVRQSHGKTLEWIGGINPNNGGTTYNQNFKGKATLTVEKSSTTAYLELRSLTSEDSALYYCARKEFSDGYYFFAYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 16 - protein, 238 aa, Artificial Sequence
  89M5 Light chain amino acid sequence
  SIGNAL (1)..(24): predicted signal sequence
MGFKMESQIQAFVFVFLWLSGVDGDIVMTQSHKFMSTSVGDRVNITCKASQDVIFAVAWYQQKPGQSPKLLIYWASTRHTGVPDRFTGSVSGTDYTLTISSVQAEDLALYYCQQHYSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 17 - protein, 214 aa, Artificial Sequence
  89M5 Light chain amino acid sequence without predicted signal sequence
DIVMTQSHKFMSTSVGDRVNITCKASQDVIFAVAWYQQKPGQSPKLLIYWASTRHTGVPDRFTGSVSGTDYTLTISSVQAEDLALYYCQQHYSTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 18 - DNA, 363 bp, Artificial Sequence
  89M5 Heavy chain variable region nucleotide sequence
gaggtccagctgcaacagtctggacctgagctggtgaagcctggggcttcagtgaagatatcctgcaagacttctggatacacattcactggatacaccatgcactgggtgaggcagagccatggaaagacccttgagtggattggaggtattaatcctaacaatggtggtactacttacaaccagaacttcaagggcaaggccacattgactgtagagaagtcctccaccacagcctacttggagctccgcagcctgacatctgaggattctgcactctattactgtgcaagaaaggagttctctgatggttactacttttttgcttactggggccaagggactctggtcactgtctctgca

SEQ ID NO: 19 - DNA, 321 bp, Artificial Sequence
  89M5 Light chain variable region nucleotide sequence
gacattgtgatgacccagtctcacaaattcatgtccacatcagtgggagacagggtcaacatcacctgcaaggccagtcaggatgtgatttttgctgtagcctggtatcaacagaaaccaggacaatctcctaaactactgatttactgggcatccacccggcacactggagtccctgatcgcttcacaggcagtgtatctgggacagattatactctcaccatcagcagtgtgcaggctgaagacctggcactttattactgtcagcaacattatagcactccgtggacgttcggtggaggcaccaagctggaaatcaaa

SEQ ID NO: 20 - DNA, 1398 bp, Artificial Sequence
  89M5 Heavy chain nucleotide sequence
atgggatggagctggatctttctctttctcctgtcaggaactgcaggtgtcctctctgaggtccagctgcaacagtctggacctgagctggtgaagcctggggcttcagtgaagatatcctgcaagacttctggatacacattcactggatacaccatgcactgggtgaggcagagccatggaaagacccttgagtggattggaggtattaatcctaacaatggtggtactacttacaaccagaacttcaagggcaaggccacattgactgtagagaagtcctccaccacagcctacttggagctccgcagcctgacatctgaggattctgcactctattactgtgcaagaaaggagttctctgatggttactacttttttgcttactggggccaagggactctggtcactgtctcttcagccaaaacgacacccccatctgtctatccactggcccctggatctgctgcccaaactaactccatggtgaccctgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatgataa

SEQ ID NO: 21 - DNA, 720 bp, Artificial Sequence
  89M5 Light chain nucleotide sequence
atgggcttcaagatggagtcacagattcaggcatttgtattcgtgtttctctggttgtctggtgttgacggagacattgtgatgacccagtctcacaaattcatgtccacatcagtgggagacagggtcaacatcacctgcaaggccagtcaggatgtgatttttgctgtagcctggtatcaacagaaaccaggacaatctcctaaactactgatttactgggcatccacccggcacactggagtccctgatcgcttcacaggcagtgtatctgggacagattatactctcaccatcagcagtgtgcaggctgaagacctggcactttattactgtcagcaacattatagcactccgtggacgttcggtggaggcaccaagctggaaatcaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttagtga

SEQ ID NO: 22 - protein, 121 aa, Artificial Sequence
  Humanized h89M5-H2L2  Heavy chain variable region amino acid sequence
QVQLVQSGAEVKKPGASVKVSCKTSGYTFTGYTMHWVRQAPGQRLEWMGGINPNNGGTTYNQNFKGRVTITRDTSASTAYMELSSLRSEDTAVYYCARKEFSDGYYFFAYWGQGTLVTVSS

SEQ ID NO: 23 - protein, 107 aa, Artificial Sequence
  h89M5-H2L2  Light chain variable region amino acid sequence
DIQMTQSPSSLSASVGDRVTITCKASQDVIFAVAWYQQKPGKAPKLLIYWASTRHTGVPSRFSGSGSGTDYTLTISSLQPEDFATYYCQQHYSTPWTFGGGTKVEIK

SEQ ID NO: 24 - protein, 466 aa, Artificial Sequence
  h89M5-H2L2  Heavy chain amino acid sequence
  SIGNAL (1)..(19): predicted signal sequence
MDWTWRILFLVAAATGAHSQVQLVQSGAEVKKPGASVKVSCKTSGYTFTGYTMHWVRQAPGQRLEWMGGINPNNGGTTYNQNFKGRVTITRDTSASTAYMELSSLRSEDTAVYYCARKEFSDGYYFFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 25 - protein, 447 aa, Artificial Sequence
  h89M5-H2L2  Heavy chain amino acid sequence without predicted signal sequence
QVQLVQSGAEVKKPGASVKVSCKTSGYTFTGYTMHWVRQAPGQRLEWMGGINPNNGGTTYNQNFKGRVTITRDTSASTAYMELSSLRSEDTAVYYCARKEFSDGYYFFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 26 - protein, 236 aa, Artificial Sequence
  h89M5-H2L2  Light chain amino acid sequence
  SIGNAL (1)..(22): predicted signal sequence
MDMRVPAQLLGLLLLWLRGARCDIQMTQSPSSLSASVGDRVTITCKASQDVIFAVAWYQQKPGKAPKLLIYWASTRHTGVPSRFSGSGSGTDYTLTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 27 - protein, 214 aa, Artificial Sequence
  h89M5-H2L2  Light chain amino acid sequence without predicted signal sequence
DIQMTQSPSSLSASVGDRVTITCKASQDVIFAVAWYQQKPGKAPKLLIYWASTRHTGVPSRFSGSGSGTDYTLTISSLQPEDFATYYCQQHYSTPWTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 28 - DNA, 363 bp, Artificial Sequence
  h89M5-H2L2  Heavy chain variable region nucleotide sequence
caggtccagctcgtgcagtctggggctgaggtgaagaagcctggggcctctgtgaaggtttcctgcaagacttctggatacaccttcactggatacaccatgcactgggttagacaggcccccggacaaaggctggagtggatgggaggtattaatcctaacaatggtggtactacttacaaccagaacttcaagggcagagtcaccattaccagggacacatccgcaagcacagcctacatggagctgtccagcctgagatctgaagacacagctgtgtattactgtgcaagaaaggagttctctgatggatactacttttttgcttactggggccaagggaccctggtcaccgtcagctca

SEQ ID NO: 29 - DNA, 321 bp, Artificial Sequence
  h89M5-H2L2  Light chain variable region nucleotide sequence
gacatccagatgacccagtctccatcctccctgtctgcatctgtcggagacagagtcaccatcacttgcaaggcctcccaggatgtgatttttgctgttgcctggtatcagcagaaaccagggaaagcccctaagctcctgatctattgggcatccacccggcacactggggtcccatcaaggttcagtggcagtggatctgggacagattacactctcaccatcagcagtctgcaacctgaagattttgcaacttactactgtcagcaacattatagcactccttggactttcggcggagggaccaaggtggagatcaaa

SEQ ID NO: 30 - DNA, 1401 bp, Artificial Sequence
  h89M5-H2L2  Heavy chain nucleotide sequence
atggactggacctggaggatactctttctcgtggcagcagccacaggagcccactcccaggtccagctcgtgcagtctggggctgaggtgaagaagcctggggcctctgtgaaggtttcctgcaagacttctggatacaccttcactggatacaccatgcactgggttagacaggcccccggacaaaggctggagtggatgggaggtattaatcctaacaatggtggtactacttacaaccagaacttcaagggcagagtcaccattaccagggacacatccgcaagcacagcctacatggagctgtccagcctgagatctgaagacacagctgtgtattactgtgcaagaaaggagttctctgatggatactacttttttgcttactggggccaagggaccctggtcaccgtcagctcagccagcacaaagggccctagcgtcttccctctggctccctgcagcaggagcaccagcgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 31 - DNA, 711 bp, Artificial Sequence
  h89M5-H2L2  Light chain nucleotide sequence
atggacatgagggtccccgcacagctcctggggctcctgctcctctggctccggggtgccagatgtgacatccagatgacccagtctccatcctccctgtctgcatctgtcggagacagagtcaccatcacttgcaaggcctcccaggatgtgatttttgctgttgcctggtatcagcagaaaccagggaaagcccctaagctcctgatctattgggcatccacccggcacactggggtcccatcaaggttcagtggcagtggatctgggacagattacactctcaccatcagcagtctgcaacctgaagattttgcaacttactactgtcagcaacattatagcactccttggactttcggcggagggaccaaggtggagatcaaacggactgtggctgcaccatctgtcttcatcttccctccatctgatgagcagttgaaatctggaactgcctctgttgtgtgcctgctgaataacttctatcccagagaggccaaagtccagtggaaggtggataacgccctccaatccggtaactcccaggagagtgtcacagagcaggacagcaaggacagcacctacagcctcagcaacaccctgacactgagcaaagcagactacgagaaacacaaagtctacgcctgcgaagtcacccatcagggcctgagctcccccgtcacaaagagcttcaacaggggagagtgctaa

SEQ ID NO: 32 - protein, 121 aa, Artificial Sequence
  h89M5-H8L5  Heavy chain variable region amino acid sequence
EVQLVQSGAEVKKPGESLRISCKGSGYSFTGYTMHWVRQMPGKGLEWMGGINPNNGGTTYNQNFKGHVTISADKSISTAYLQWSSLKASDTAMYYCARKEFSDGYYFFAYWGQGTLVTVSS

SEQ ID NO: 33 - protein, 107 aa, Artificial Sequence
  h89M5-H8L5  Light chain variable region amino acid sequence
DIVMTQSPDSLAVSLGERATINCKASQDVIFAVAWYQQKPGQPPKLLIYWASTRHTGVPDRFSGSGSGTDFTLTISSLQAEDVAVYYCQQHYSTPWTFGGGTKVEIK

SEQ ID NO: 34 - protein, 470 aa, Artificial Sequence
  h89M5-H8L5  Heavy chain amino acid sequence
  SIGNAL (1)..(19): predicted signal sequence
MDWTWRILFLVAAATGAHSEVQLVQSGAEVKKPGESLRISCKGSGYSFTGYTMHWVRQMPGKGLEWMGGINPNNGGTTYNQNFKGHVTISADKSISTAYLQWSSLKASDTAMYYCARKEFSDGYYFFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 451 aa, Artificial Sequence
  h89M5-H8L5  Heavy chain amino acid sequence without predicted signal sequence
EVQLVQSGAEVKKPGESLRISCKGSGYSFTGYTMHWVRQMPGKGLEWMGGINPNNGGTTYNQNFKGHVTISADKSISTAYLQWSSLKASDTAMYYCARKEFSDGYYFFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 234 aa, Artificial Sequence
  h89M5-H8L5  Light chain amino acid sequence
  SIGNAL (1)..(20): predicted signal sequence
MVLQTQVFISLLLWISGAYGDIVMTQSPDSLAVSLGERATINCKASQDVIFAVAWYQQKPGQPPKLLIYWASTRHTGVPDRFSGSGSGTDFTLTISSLQAEDVAVYYCQQHYSTPWTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 37 - protein, 214 aa, Artificial Sequence
  h89M5-H8L5  Light chain amino acid sequence without predicted signal sequence
DIVMTQSPDSLAVSLGERATINCKASQDVIFAVAWYQQKPGQPPKLLIYWASTRHTGVPDRFSGSGSGTDFTLTISSLQAEDVAVYYCQQHYSTPWTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 38 - DNA, 363 bp, Artificial Sequence
  h89M5-H8L5  Heavy chain variable region nucleotide sequence
gaagtgcagctggtgcagtctggagcagaggtcaaaaagcccggggagtctctgaggatctcctgcaagggttctggatacagctttactggatacaccatgcactgggtgcgccagatgcccgggaaaggactggagtggatggggggtattaatcctaacaatggtggtactacttacaaccagaacttcaagggccacgtcaccatctcagctgacaagtccatcagcactgcctacctgcaatggagcagcctgaaggcttctgacaccgccatgtattactgtgcaagaaaggagttctctgatggatactacttttttgcttactggggccaagggaccctggtgaccgtcagctca

SEQ ID NO: 39 - DNA, 321 bp, Artificial Sequence
  h89M5-H8L5  Light chain variable region nucleotide sequence
gacatcgtgatgacccagtctccagactccctggctgtgtctctgggcgagagggccaccatcaactgcaaggcttcccaggacgtgatttttgctgttgcctggtatcagcagaaaccaggacagcctcctaagctgctcatttactgggcatctacccgccacactggggtccctgaccgcttcagtggcagcgggtctgggacagatttcactctcaccatcagcagcctccaggctgaagatgtggcagtttattactgtcagcaacattatagcactccttggactttcggcggagggaccaaggtggagatcaaa

SEQ ID NO: 40 - DNA, 1413 bp, Artificial Sequence
  h89M5-H8L5  Heavy chain nucleotide sequence
atggactggacctggaggatactctttctcgtggcggcagccacaggagcccactccgaagtgcagctggtgcagtctggagcagaggtcaaaaagcccggggagtctctgaggatctcctgcaagggttctggatacagctttactggatacaccatgcactgggtgcgccagatgcccgggaaaggactggagtggatggggggtattaatcctaacaatggtggtactacttacaaccagaacttcaagggccacgtcaccatctcagctgacaagtccatcagcactgcctacctgcaatggagcagcctgaaggcttctgacaccgccatgtattactgtgcaagaaaggagttctctgatggatactacttttttgcttactggggccaagggaccctggtgaccgtcagctcagccagcacaaagggcccctccgtgttccctctggccccttcctccaagtccacctccggcggcaccgccgctctgggctgcctggtgaaggactacttccctgagcctgtgaccgtgtcctggaactctggcgcactgacctctggcgtgcacaccttcccagccgtgctccagtcctccggcctgtactccctgtcctccgtcgtcaccgtgccttcctcctccctgggcacccagacctacatctgcaacgtgaaccacaagccttccaacacaaaggtggacaagcgggtggagcctaagtcctgcgacaagacccacacctgccctccctgccctgcccctgagctgctgggcggaccttccgtgttcctgttccctcctaagcctaaggacaccctgatgatctcccggacccctgaagtgacatgcgtggtggtggacgtgtcccacgaggaccctgaggtgaagttcaactggtatgtggacggcgtggaggtgcacaacgctaagaccaagccgagggaggagcagtacaactccacctaccgggtggtgtctgtgctgaccgtgctgcaccaggactggctgaacggcaaagaatacaagtgcaaggtctccaacaaggccctgcccgctcccatcgagaaaaccatcagcaaggcaaagggccagcctcgcgagcctcaggtgtacaccctgccacccagccgggaggagatgaccaagaaccaggtgtccctgacctgtctggtgaagggcttttacccttccgatattgccgtggagtgggagtctaacggccagcccgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagagcctgtctctgtctcctggcaagtga

SEQ ID NO: 41 - DNA, 705 bp, Artificial Sequence
  h89M5-H8L5  Light chain nucleotide sequence
atggtgctccagacccaggtcttcatttctctgctcctctggatctctggtgcctacggggacatcgtgatgacccagtctccagactccctggctgtgtctctgggcgagagggccaccatcaactgcaaggcttcccaggacgtgatttttgctgttgcctggtatcagcagaaaccaggacagcctcctaagctgctcatttactgggcatctacccgccacactggggtccctgaccgcttcagtggcagcgggtctgggacagatttcactctcaccatcagcagcctccaggctgaagatgtggcagtttattactgtcagcaacattatagcactccttggactttcggcggagggaccaaggtggagatcaaacggactgtggctgcaccatctgtcttcatcttccctccatctgatgagcagttgaaatctggaactgcctctgttgtgtgcctgctgaataacttctatcccagagaggccaaagtccagtggaaggtggataacgccctccaatccggtaactcccaggagagtgtcacagagcaggacagcaaggacagcacctacagcctcagcaacaccctgacactgagcaaagcagactacgagaaacacaaagtctatgcctgcgaagtcacccatcagggcctgtcttcccccgtcacaaagagcttcaacaggggagagtgctaa